(19) United States
(12) Patent Application Publication
(10) Pub. No.: US 2005/0215571 A1
(43) Pub. Date: Sep. 29, 2005

(54) Therapeutic combination for cognititon enhancement and psychotic disorders

(75) Inventor: Steve Joseph Romano, New York, NY (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/018,100

(22) Filed: Dec. 20, 2004

Related U.S. Application Data
(60) Provisional application No. 60/532,082, filed on Dec. 23, 2003.

(51) Int. Cl. A61K 31/519; A61K 31/473

(52) U.S. Cl. 514/259.41; 514/295

(57) ABSTRACT

This invention relates to combinations of an atypical antipsychotic, and a nicotinic receptor agonist or antagonist, kits containing such combinations, pharmaceutical compositions comprising such combinations, and methods of using such combinations to treat patients suffering from cognitive impairment disorders or psychotic disorders or conditions.

This application claims priority under 35 USC 119 of U.S. Provisional Application 60/532,082, filed Dec. 23, 2003. The entire content of the prior application, U.S. Provisional Application 60/532,082, is incorporated herein by reference.

### FIELD OF THE INVENTION

The present invention relates to pharmaceutical compositions comprising combinations of ziprasidone, a prodrug, thereof a pharmaceutically acceptable salt thereof, or a pharmaceutically acceptable salt of said prodrug, and a nicotinic receptor agonist or antagonist; kits comprising such combinations; and methods of using such combinations to treat patients, including humans, suffering from cognitive impairment, or psychotic disorders or conditions. This invention also relates to additive and synergistic combinations of ziprasidone, a prodrug thereof, a pharmaceutically acceptable salt thereof, or a pharmaceutically acceptable salt of said prodrug and a nicotinic receptor agonist or antagonist, which additive and synergistic combinations are useful in treating patients, including humans, suffering from cognitive impairment, or psychotic disorders or conditions.

### BACKGROUND OF THE INVENTION

Schizophrenia is a common and serious mental disorder characterized by loss of contact with reality (psychosis), hallucinations (false perceptions), delusions (false beliefs), abnormal thinking, flattened affect, diminished motivation, and disturbed work and social functioning.

Atypical antipsychotics offer several clinical benefits over the conventional antipsychotics, which were the mainstays of care until the past decade. The principal mechanism, underlying the many clinical benefits of the atypical agents is separating the antipsychotic effect from the extrapyramidal side effects (EPS). The distinct advantages over traditional antipsychotic medications include greater improvement in negative symptoms, such as social withdrawal, and lower risk of Parkinsonian side effects and tardive dyskinesia.

The conventional antipsychotics are antagonists of dopamine (D₂) receptors. The atypical antipsychotics likewise have D₂ antagonistic properties, but possess different binding kinetics to these receptors and activity at other receptors, particularly 5-HT_{2A}, 5-HT_{2c} and 5-HT_{1D} (Schmidt B et al, Soc. Neurosci. Abstr., 24:2177; (1998)).

Examples of atypical antipsychotics include clozapine (Clozaril®), risperidone (Risperdal®), olanzapine (Zyprexa®), quetiapine (Seroquel®), aripiprazole (Abilify®), and ziprasidone (Geodon®). Ziprasidone is an atypical antipsychotic whose efficacy in the treatment of schizophrenia has been examined in an extensive clinical trial program that includes both short term and long term studies. Ziprasidone is indicated for the treatment of schizophrenia. Cognitive function is strongly associated with patient outcome in schizophrenia. Efficacy for ziprasidone in improving cognition in schizophrenic patients has been confirmed in cognitive battery tests such as the PANSS cognitive subscale (Harvey et al., Cognitive, affective, and prosocial improvement after switch to ziprasidone, American Psychiatric Association Annual Meeting, San Francisco Calif., May 17-21, 2003). Improvements in cognitive function in the following subscale parameters have been confirmed with ziprasidone treatment: difficulty in abstract thinking, stereotyped thinking, tension, mannerisms and posturing, poor attention, and lack or judgment and insight.

Both Alzheimer's and non-Alzheimer's dementias can be accompanied by psychosis. A beneficial effect of ziprasidone in dementia-related psychosis has been demonstrated in elderly patients (Berkowitz et al., Ziprasidone for elderly dementia: Case series, American Psychiatric Association Meeting, San Francisco, Calif., May 17-21, 2003).

U.S. Pat. Nos. 4,831,031 and 4,883,795, which are hereby incorporated in their entireties by reference, each respectively disclose that ziprasidone has utility in the treatment of psychotic disorders. U.S. Pat. Nos. 6,245,766 and 6,126,373, which are hereby incorporated in their entireties by reference, each disclose that ziprasidone has utility in the treatment of cognition impairment and mood disorders.

Disorders of cognition are generally characterized by one or more mental symptoms such as forgetfulness, confusion, memory loss, attentional deficits or affective or emotional disturbances. These symptoms may arise as a result of the natural aging process or from organic brain disease, cerebrovascular disease, head injury or developmental or genetic defects. Although cognitive disorders often accompany the general aging process, presenile and senile primary degenerative dementia are the most commonly accepted causes of mental deterioration in the elderly.

Studies in both human and experimental animals suggest that nicotine has cognition-enhancing properties. Evidence in the literature suggests that nicotine may improve attentiveness (Levin, E.; 108 Psychopharm. 417-431, (1992)). In animal studies, nicotine can reverse deficits in working memory in brain-lesioned rats (Levin et al., 1 Cognitive Brain Research, 137-143, (1993)) and also improves performance on serial choice tasks, which are thought to partially model symptoms of Attention Deficit Hyperactivity Disorder (Muir, et al., 118 Psychopharm., 82-92; (1995)).

Nicotinic acetylcholine receptors are found in the autonomic nervous system, the neuromuscular junction and the brain in vertebrates. It is known that nicotine receptors are present in significant numbers in the brain, and their involvement in higher functions, such as learning and memory, has been recognized. Nicotinic receptor agonists or antagonists have been disclosed to be useful for neurological and mental disorders including cognitive impairment disorders such as Alzheimer's disease. It has been observed that nicotinic acetylcholine receptors, which bind nicotine and other nicotinic agonists with high affinity, are depleted during the progression of Alzheimer's disease (Giacobini, 27 J. Neurosci. Res., 548, (1990); Baron, 36 Neurology, 1490; (1986); Nordberg et al., 72 J. Neurosci. Lett., 115-119; (1986)). Further, in animal studies using open field habituation learning, nicotine administration into the nucleus accumbens enhanced behavioral habituation indicating a facilitation of memory (Schildein, S. et al., 77 Neurobio. Learn. Memory, 277-90; (2002)).

In addition to the role of nicotine agonists, evidence also exists that nicotine antagonism may also play an important role in neuropsychiatric disorders. Depression may be mediated through excessive nicotinic receptor activation and the therapeutic action of antidepressants may, in part, be mediated through nicotine receptors (Shyle, R D et al, 7 Mol. Psych., 525-35; (2002)). For example, the nicotinic receptor antagonist, mecamylamine, has been found to reduce the symptoms of depression and mood disorders (Williams et al, 7 Drug News & Perspect., 205-223; (1994)).

Central cholinergic neurotransmission involves two major receptor subtypes: muscarinic and nicotinic. The cholinergic hypothesis (Bartus, et al., 217 Science, 408; (1982)) states that the enzyme choline acetyltransferase is depleted in Alzheimer's disease. Depletion of this enzyme prevents the conversion of choline to acetylcholine. The post-synaptic receptors for the most part remain unimpaired. A chemical replacement for acetylcholine, e.g., a nicotinic or a muscarinic agonist would be effective only if the receptor remains intact.

Nicotine has been suggested to possess an ability to activate nicotinic cholinergic receptors upon acute administration, and to elicit an increase in the number of such receptors upon chronic administration to animals (Rowell, 31 Adv. Behav. Biol., 191; (1987); Marks, J., 226 Pharmacol. Exp. Ther, 817; (1983)). It also has been proposed that nicotine can act directly to elicit the release of acetylcholine in brain tissue, to improve cognitive functions, and to enhance attention (Rowell, et al., 43 J. Neurochem., 1593; (1984); Sherwood, 8 Human Psychopharm., 155-184; (1993); Hodges, et al., Bio. of Nicotine., Lippiello, et al. (ed), 157; (1991); Sahakian, et al., 154; Br. J. Psych., 797; (1989); and U.S. Pat. No. 4,965,074 to Leeson and U.S. Pat. No. 5,242,935 to Lippiello et al.). Methods for treating Alzheimer's disease have been proposed, including those in U.S. Pat. No. 5,212,188 to Caldwell et al. and U.S. Pat. No. 5,227,391 to Caldwell et al. and European Patent Application No. 588,917.

Impaired attention may also be a characteristic of Alzheimer's disease, although Alzheimer's patients typically remain alert (Coyle et al., Alzheimer's Disease: A Disorder of Cholinergic Innervation, Science 219:1184-90; (1983)), and the underlying disorders and known treatments are very different (Grady, C. L. et al. J. Clin. Exp. Neuropsychology, 10:576; (1988)). Alzheimer's disease involves progressive and profound loss of memory, postulated to involve a deficiency in brain cortical acetylcholine affecting cholinergic synapses. This deficiency is thought to be caused by selective degeneration of acetylcholine-releasing neurons (Coyle, supra).

Certain synapses of the brain use acetylcholine as a neural transmitter, to transmit messages across the synapse to a cholinergic receptor. During normal transmission, acetylcholine crosses the synaptic gap to carry the message by stimulating the cholinergic receptor. Memory is thought to be related, at least in part, to post-synaptic changes which occur as a result of the timing and strength of acetylcholine stimulation during learning, with certain experiences tending to block or facilitate corresponding neural pathways, i.e. making it more or less difficult to stimulate the same post-synaptic receptor at a future time. (Deutsch, The Cholinergic Synapse and the Site of Memory; Science 174:788-94; (1971)). Acetylcholine also is rapidly destroyed by the enzyme cholinesterase. Thus, insufficient acetylcholine or excess cholinesterase can interfere with synaptic transmission by too rapidly-destroying the acetylcholine message, resulting in weak cholinergic stimulation, which can be experienced as memory loss. When this condition is chronic, i.e. from degeneration of acetylcholine-releasing neurons, Alzheimer's syndrome may develop.

U.S. Pat. Nos. 5,977,131 and 6,020,335, which are hereby incorporated by reference, each disclose nicotinic receptor agonists or antagonists with utility in the treatment of cognitive impairment from dementia and Alzheimer's disease. Additionally, varenicline is a partial nicotine receptor agonist for reducing the symptoms of nicotine withdrawal and the satisfaction associated with smoking and for the treatment of psychosis and schizophrenia. The following commonly assigned patents and applications pertain to varenicline: WO 99/35131, U.S. Pat. No. 6,410,550, Patent Appln. Nos. 1997070245, 2002072524, 2002072525, 2002111350, and 2002132824, and are herein incorporated by reference in their entireties. Other compounds that bind to neuronal nicotinic receptor sites are referred to in U.S. Pat. No. 6,020,335. The foregoing patent is owned in common with the present application, and is incorporated herein by reference in its entirety.

The effectiveness of nicotine in treating various psychological conditions has been recognized in U.S. Pat. Nos. 5,187,169 and 5,298,257. Nicotine has been found to potentiate the behavioral effects of neuroleptics such as haloperidol while diminishing the side effect profile. Clinical trials have indicated that both nicotine gum and nicotine patches can ameliorate the symptoms of Tourette's syndrome in adolescents not satisfactorily controlled with neuroleptics (Decker, M. et al, Neuronal Nicotinic Acetylcholine Receptors: Novel Targets for CNS Therapeutics, Amer. Coll. Neuropsychiat.; (1990)). As described in U.S. Pat. No. 5,889,029, tests using human and animal tissue show that cotinine, a metabolite of nicotine, has the same high affinity for many of the same receptor sites as clozapine. Consequently, its action and effectiveness in schizophrenia is believed to be of a similar origin.

Nicotine had been found to be effective in normalizing the psychophysiological defects of schizophrenia. It is well known that schizophrenics are heavy smokers. Among psychiatric patients, those with schizophrenia are more likely to be smokers than those with other psychiatric diagnoses. This finding supports an explanation for the consumption of nicotine by these patients as a self-administered therapy.

Cholinergic neurons may be involved in schizophrenia. Bungarotoxin is a selective nicotine antagonist isolated from the venom of a Taiwanese snake which is a potent inhibitor of the acetylcholine release at the neuromuscular junction. It has been shown that bungarotoxin-sensitive cholinergic receptors in the hippocampus are involved in duplicating a second sonic response characteristic of schizophrenia. Nicotine appears to be effective in inhibiting typical schizophrenic activity when used in combination with mecamylamine (MEC). The use of nicotine is not, however, acceptable as a therapy for schizophrenia because a high dose of nicotine (which can be toxic) is needed and the effect is short-lived. Thus, tachyphylaxis occurs in short order (Freedman R., et al., 38 Biol. Psy., 22-33; (1995)).

The nicotine receptor antagonist mecamylamine has demonstrated efficacy in treating a range of neuropsychiatric disorders such as mood disorders and bipolar disorders (Silver A, et al, 18 Today's Ther. Trends, 255-273; (2000)).

As set forth below, various nicotine compounds and their uses are known. For example, U.S. Pat. No. 4,965,074 to Leeson discloses a nicotine derivative compound for the treatment of senile dementia and Alzheimer's type diseases.

U.S. Pat. No. 5,278,176 to Lin is directed to nicotine receptor agonists. These compounds are useful for attentional hyperactivity disorder, and anxiety associated cognitive impairment or substance abuse withdrawal.

U.S. Pat. No. 5,776,957 to Crooks discloses use of an isolated enantiomer of nornicotine for treating Alzheimer's disease and schizophrenia. Nornicotine is an alkaloid, C₉H₁₂N₂, extracted from tobacco and related to nicotine but having a lower toxicity.

U.S. Pat. No. 5,276,043 to Lippiello et al. is directed to nicotine derivatives useful for the treatment of neurodegenerative diseases.

U.S. Pat. No. 5,227,391 to Caldwell et al. is directed to an R-(+) nicotine compound. U.S. Pat. No. 5,214,060 to Caldwell et al. discloses compounds for the treatment of neurodegenerative diseases.

U.S. Pat. No. 5,242,934 to Lippiello et al. is directed to gamma nicotine compounds for the treatment of neurodegenerative diseases. U.S. Pat. No. 5,223,497 to Gawin et al. is directed to compounds for treating habit disorders. U.S. Pat. No. 5,278,045 to Tam discloses nicotine compounds for the enhancement of dopaminergic function.

U.S. Pat. No. 5,232,933 to Lippiello et al. discloses α-nicotine compounds for the treatment of neurodegenerative diseases. U.S. Pat. No. 5,138,062 to Osdene et al. discloses nicotine compounds. U.S. Pat. No. 4,966,916 to Abood discloses agonists and antagonists to nicotine as smoking deterrents.

According to the DSM-IV, dementia is characterized by multiple cognitive defects that include impairment in memory. The psychotic symptoms associated with dementia are treated with antipsychotic agents as add-on therapy to cognition enhancement therapy. Therefore, a combination product would have utility in this patient population.

There is a need in the art for new and improved treatments for other diseases, disorders, and syndromes that are characterized by symptoms of cognitive impairment and/or psychotic disorders or conditions.

Mental illness is particularly difficult to treat in that not all patients react similarly to the same treatment regimen. Patients often require multiple drug therapies. There also exists a large number of untreated individuals and treatment-resistant patients in need of effective therapy.

Exacerbating this is the problem of patient noncompliance. For example, it is conventionally thought that substantial numbers of patients with mental illnesses are not compliant or only partially compliant with their medication. Poor compliance can cause relapses thereby negating whatever benefits were achieved through treatment in the first place.

Simplification of the regimen by combining several therapeutic agents, reduces the opportunity for patient noncompliance as occurs with a more rigorous schedule. There is, therefore, a need for pharmaceutical combinations and pharmaceutical kits which employ atypical antipsychotics efficacious for the treatment of, e.g. cognitive impairment or psychotic disorders and conditions.

The present invention is directed to compositions which reduce or overcome these disadvantages. More particularly, this invention provides novel pharmaceutical combinations of atypical antipsychotics and nicotinic receptor agonists or antagonists for the treatment of cognitive impairment and psychotic disorders and symptoms.

### SUMMARY OF THE INVENTION

The present invention is directed to pharmaceutical compositions, therapeutic methods of treatment, and kits which employ an atypical antipsychotic together with a nicotinic receptor agonist or antagonist.

According to the invention, these pharmaceutical combinations can provide synergistic or additive effects in treating diseases or conditions of impaired cognition or in treating psychotic disorders or conditions. These combinations can offer some or all of the following: symptomatic relief of cognitive impairment, less side effects, a reduction in use of concomitant psychotropic medications such as antidepressants, or sedatives and mood stabilizers such as lithium, and prevention of future decline in psychosis or cognitive function.

Thus, according to one aspect, the present invention provides a combination of an atypical antipsychotic agent and a nicotinic receptor agonist or antagonist. Atypical antipsychotics which can be used in the present invention include olanzapine, clozapine, risperidone, sertindole, quetiapine, aripiprazole, amisulpride and ziprasidone. In general, pharmaceutical combinations and methods of treatment using ziprasidone as the first therapeutic agent are preferred.

A further feature of the present invention is a method of reducing the amount of the atypical antipsychotic agent required to produce cognitive enhancement or an antipsychotic effect which comprises treating a patient with a therapeutically effective amount of a drug combination according to the present invention.

It is also a feature of this invention that the use of such drug combinations will enhance the effect of the atypical antipsychotic agent to be used and therefore allow reduced quantities of the antipsychotic agent to be used and, therefore allow better management of drug-related toxicity and side effects.

The invention offers advantages over previous methods for treating neuropsychiatric disorders. The method of treatment of the present invention will enhance the effect of the nicotinic receptor agonist or antagonist used and therefore permit reduced quantities of the nicotinic receptor agonist or antagonist to be used and, therefore permit improved management of drug-related toxicity and side effects. Other features and advantages of the invention will be apparent from the following detailed description and from the claims.

### DETAILED DESCRIPTION OF THE INVENTION

The present invention is directed to a pharmaceutical composition for treatment of cognitive impairment or a psychotic disorder in a mammal, including a human, comprising (a) an amount of an atypical antipsychotic, a prodrug thereof, or a pharmaceutically acceptable salt thereof, or a pharmaceutically acceptable salt of said prodrug; and (b) an amount of a nicotinic receptor agonist or antagonist, and a pharmaceutically acceptable carrier, wherein the amounts (a) and (b) are together effective in treating said cognitive impairment or psychotic disorder.

The present invention is further directed to a method for treating cognitive impairment or a psychotic disorder in a mammal, including a human, which method comprises administering (a) an amount of an atypical antipsychotic; and (b) an amount of a nicotinic receptor agonist or antagonist to said mammal, wherein the amounts (a) and (b) are together effective in treating said cognitive impairment or psychotic disorder.

In one embodiment, the present invention is directed to a method of treating cognitive impairment or a psychotic disorder in a mammal, including a human, which method comprises administering (a) an amount of ziprasidone, a prodrug thereof or a pharmaceutically acceptable salt of ziprasidone or said prodrug, and (b) an amount of a nicotinic receptor agonist or antagonist to said mammal; wherein the amounts of (a) and (b) are together effective in treating said cognitive impairment or psychotic disorder.

This invention is also directed to kits for achieving a therapeutic effect in a mammal, including a human, comprising an amount of ziprasidone, a prodrug thereof or a pharmaceutically acceptable salt of ziprasidone or said prodrug and a pharmaceutically acceptable vehicle, carrier or diluent in a first unit dosage form; and an amount of a nicotinic receptor agonist or antagonist, and a pharmaceutically acceptable vehicle, carrier or diluent in a second unit dosage form, and a container.

The methods of this invention provide therapeutic treatment of cognitive impairment and/or a psychotic disorder in a mammal, preferably a human.

“Cognitive impairment” refers to an acquired deficit in one or more of memory function, problem solving, orientation and abstraction. Examples of standard tests for measuring cognitive impairment include, the Mini Mental State Examination, the Global Deterioration Scale and Geriatric Depression Scale, the Randt Memory Test and the Alzheimer's Disease Assessment Scale.

“Cognitive impairment” which may be treated by the methods of this invention includes, inter alia, dementia, cognitive impairments caused by traumatic brain injury, Alzheimer's disease, age-related memory disorder, vascular dementia, dementia due to other general medical conditions (e.g., Human Immunodeficiency Virus disease, head trauma, Parkinson's disease, Huntington's disease), substance-induced persisting dementia (i.e., due to drug abuse, a medication, or toxin exposure), dementia due to multiple etiologies, or dementia not otherwise specified, and cognitive disorder not otherwise specified. Other conditions having associated cognitive impairment which may be treated by the methods of this invention appear in DSM-IV, 4^{th} ed., pp. 135-180.

“Dementia” refers to global deterioration of intellectual functioning in clear consciousness, and is characterized by one or more symptoms of disorientation, impaired memory, impaired judgment, and/or impaired intellect. The symptoms of “dementia” are generally worse than, and can encompass, the symptoms of “cognitive impairment.”

“Cognitive impairments caused by traumatic brain injury” refers to cognitive impairments, as defined herein, that are associated with or caused by traumatic brain injuries, and other traumas to the head, such as, for example, traumas caused by accidents and/or sports injuries.

“Cognitive impairments caused by traumatic brain injury” includes dementia pugilistica, which is severe brain damage caused by repeated blows to the head (e.g., from boxing). Dementia pugilistica is a chronic and progressive clinical syndrome characterized by neurological evidence of damage to pyramidal, extrapyramidal, and cerebellar systems with associated psychosis, dementia, personality change and impaired social functioning and/or prominent signs/symptoms of Parkinsonism (e.g., tremors, dysarthria, rigidity, bradykinesia, other extrapyramidal signs).

The methods of this invention include therapeutic treatment of psychotic disorders or conditions. Psychotic disorders which can be treated by the methods of this invention include, inter alia, schizophrenia, schizophreniform disorder, schizoaffective disorder, delusional disorder, brief psychotic disorder, and shared psychotic disorder.

Examples of nicotinic receptor agonists or antagonists for use in the combinations, pharmaceutical compositions, methods and kits of this invention include: varenicline, azaindole-ethylamine derivatives as described in U.S. Pat. No. 5,977,131, and analogs, derivatives, prodrugs, and pharmaceutically acceptable salts of the nicotinic receptor agonists or antagonists and the prodrugs.

A particularly preferred nicotinic receptor agonist for use in the combinations, pharmaceutical compositions, methods and kits of this invention is varenicline; 7,8,9,10-tetrahydro-6,10-methano-6H-pyrazino[2,3-h][3]benazepine(2R,3R)-2,3-dihydroxybutanedioate, or any pharmaceutically acceptable salt thereof, including any polymorph or any prodrug thereof, or any pharmaceutically acceptable salt of such prodrug. A preferred salt of varenicline is varenicline tartrate. Varenicline is a partial nicotine agonist with affinity for some nicotine receptor subtypes but not others. Synthesis of varenicline tartrate is disclosed in WO 99/35131, U.S. Pat. No. 6,410,550, Patent Appln. Nos. 1997070245, 2002072524, 2002072525, 2002111350, and 2002132824, which are herein incorporated by reference in their entireties.

The combinations of this invention include at least two active components: an atypical antipsychotic, a prodrug thereof, a pharmaceutically acceptable salt thereof, or a pharmaceutically acceptable salt of said prodrug, and a nicotinic receptor agonist or antagonist, a prodrug thereof or a pharmaceutically acceptable salt of the nicotinic receptor agonist or antagonist or prodrug. The combinations of this invention also include a pharmaceutically acceptable vehicle, carrier or diluent.

The combinations may result in synergistic action allowing a lower dose of the atypical antipsychotic to be administered while achieving at least the same psychotropic effect as achieved with a standard dose of the atypical antipsychotic. The dosage of the atypical antipsychotic may be reduced by about 25-90%, for example, about 40-80% and typically about 50-70%. The reduction in amount of antipsychotic required will be dependent on the amount of the second therapeutic agent given.

Another advantage of the combination is that the synergistic action allows the dose of the nicotinic receptor agonist or antagonist to be decreased thereby resulting in less side effects.

The selection of the dosage of the first and second therapeutic agents is that which can provide relief to the patient as measured by a reduction or amelioration of symptoms associated with the disorder or condition of the patient. As is well known, the dosage of each component depends on several factors such as the potency of the selected specific compound, the mode of administration, the age and weight of the patient, the severity of the condition to be treated, and the like. Determining a dose is within the skill of the ordinary artisan. To the extent necessary for completeness, the synthesis of the components of the compositions and dosages are as described in the listed patents above or the Physicians' Desk Reference, 57th ed., Thompson, 2003 which are expressly incorporated herein by reference. Desirably, when ziprasidone is selected as the active agent, the daily dose contains from about 5 mg to about 460 mg. More preferably, each dose of the first component contains about 20 mg to about 320 mg of the ziprasidone, and even more preferably, each dose contains from about 20 mg to about 160 mg of ziprasidone. Pediatric dosages may be less such as for example in the range of about 0.5 mg to about 40 mg daily. This dosage form permits the full daily dosage to be administered in one or two oral doses, for example.

General outlines of the dosages for the atypical antipsychotics, and some preferred dosages, are provided herein. This list is not intended to be complete but is merely a guideline for any of the desired combinations of the present invention.
Olanzapine: from about 0.25 to about 100 mg, once/day; preferably, from about 1 to about 30 mg, once/day; and most preferably about 1 to about 25 mg once/day;
Clozapine: from about 12.5 to about 900 mg daily; preferably, from about 150 to about 450 mg daily;
Risperidone: from about 0.25 to about 16 mg daily; preferably, from about 2-8 mg daily;
Sertindole: from about 0.0001 to about 1.0 mg/kg daily;
Quetiapine: from about 1.0 to about 40 mg/kg given once daily or in divided doses;
Asenapine: from about 0.005 to about 60 mg total per day, given as a single dose or in divided doses;
Paliperidone: from about 0.01 mg/kg to about 4 mg/kg body weight, more preferably from about 0.04 to about 2 mg/kg body weight;
Bifeprunox.

The presently preferred atypical antipsychotic used according to the invention is ziprasidone. Ziprasidone (5-[2-[4-(1,2-benzisothiazol-3-yl)piperazin-1-yl]ethyl]-6-chloroindolin-2-one) is a benzisothiazolyl piperazine atypical antipsychotic with in vitro activity as a 5-HT_{1A} receptor agonist and an inhibitor of serotonin and norepinephrine reuptake (U.S. Pat. No. 4,831,031). The postsynaptic 5-HT_{1A} receptor has been implicated in both depressive and anxiety disorders (N M Barnes, T Sharp, 38 Neuropharmacology 1083-152,1999). Oral bioavailability of ziprasidone taken with food is approximately 60%, half-life is approximately 6-7 hours, and protein binding is extensive.

Ziprasidone is efficacious for the treatment of patients with schizophrenia and schizomood disorders, refractory schizophrenia, cognitive impairment in schizophrenia, affective and anxiety symptoms associated with schizoaffective disorder and bipolar disorder. The drug is considered a safe and efficacious atypical antipsychotic (Charles Caley & Chandra Cooper, 36 Ann. Pharmacother. 839-51; (2002).

The present invention is useful in treating mental disorders and conditions, the treatment of which is facilitated by the administration of ziprasidone. Thus, the present invention has application where ziprasidone use is indicated as, e.g., in U.S. Pat. Nos. 6,245,766; 6,245,765; 6,387,904; 5,312,925; 4,831,031; and European EP 0901789 published Mar. 17, 1999, all of which are incorporated herein by reference.

Other atypical antipsychotics which can be used include, but are not limited to: Olanzapine, 2-methyl-4-(4-methyl-1-piperazinyl)-10H-thieno[2,3-b][1,5]benzodiazepine. Olanizapine is a known compound and is described in U.S. Pat. No. 5,229,382 as being useful for the treatment of schizophrenia, schizophreniform disorder, acute mania, mild anxiety states, and psychosis. U.S. Pat. No. 5,229,382 is herein incorporated herein by reference in its entirety;
Clozapine, 8-chloro-11-(4-methyl-1-piperazinyl)-5H-dibenzo[b,e][1,4]diazepine. Clozapine is described in U.S. Pat. No. 3,539,573, which is herein incorporated by reference in its entirety. Clinical efficacy in the treatment of schizophrenia is described (Hanes, et al., Psychopharmacol. Bull., 24, 62 (1988));
Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one. Risperidone and its use in the treatment of psychotic diseases are described in U.S. Pat. No. 4,804,663, which is herein incorporated by reference in its entirety;
Sertindole, 1-[2-[4-[5-chloro-1-(4-fluorophenyl)-1H-indol-3-yl]-1-piperidinyl]ethyl]imidazolidin-2-one. Sertindole is described in U.S. Pat. No. 4,710,500. Its use in the treatment of schizophrenia is described in U.S. Pat. Nos. 5,112,838 and 5,238,945. U.S. Pat. Nos. 4,710,500; 5,112,838; and 5,238,945 are herein incorporated by reference in their entireties;
Quetiapine, 5-[2-(4-dibenzo[b,f][1,4]thiazepin-11-yl-1-piperazinyl)ethoxy]ethanol. Quetiapine and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt.

Aripiprazole, 7-{4-[4-(2,3-dichlorophenyl)-1-piperazinyl]-butoxy}-3,4-dihydro carbostyril or 7-{4-[4-(2,3-dichlorophenyl)-1-piperazinyl]-butoxy}-3,4-dihydro-2(1H)-quinolinone. Aripiprazole is an atypical antipsychotic agent used for the treatment of schizophrenia and described in U.S. Pat. No. 4,734,416 and U.S. Pat. No. 5,006,528, which are herein incorporated by reference in their entireties.

Amisuipride, which is described in U.S. Pat. No. 4,401,822. U.S. Pat. No. 4,401,822 is incorporated herein in its entirety.

Asenapine, trans-5-chloro-2-methyl-2,3,3a,12b-tetrahydro-1H-dibenz[2,3:6,7]oxepino[4,5-c]pyrrole. Preparation and use of asenapine is described in U.S. Pat. Nos. 4,145,434 and 5,763,476, the entire contents of which are incorporated herein by reference.

Paliperidone, 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one. Preparation and use of paliparidone is described, for example, in U.S. Pat. Nos. 6,320,048; 5,158,952; and 5,254,556, the entire contents of which are incorporated herein by reference.

Bifeprunox, 2-[4-[4-(5-fluoro-1H-indol-3-yl)-3,6-dihydro-1 (2H)-pyridinyl]butyl]-1H-isoindole-1,3(2H)-dione. Preparation and use of bifeprunox is described in U.S. Pat. No. 6,225,312, which is incorporated in its entirety herein.

A preferred combination is ziprasidone with a nicotinic receptor agonist or antagonist.

The term “nicotinic receptor agonist”, where used in the description and the claims, is synonymous with the term “nicotine agonist”. These terms are used interchangeably throughout the description and claims. Likewise, the terms “nictonic receptor antagonist” and “nicotine antagonist” are synonymous herein and are herein used interchangeably. The term “nicotinic agonist” includes nicotinic receptor partial agonists, and nicotinic receptor full agonists, and the term “nicotinic receptor antagonist” includes nicotinic receptor partial antagonists and nicotinic receptor full antagonists.

The term “nicotinic agonist” and “nicotine receptor agonist” refer to a compound, which produces the physiological responses associated with nicotinic cholinergic activation. Nicotinic agonists interact with nicotinic receptor binding sites.

The terms “nicotinic antagonists” and “nicotine receptor antagonists” refer to both competitive nicotinic receptor antagonists and non-competitive nicotinic antagonists.

By “competitive nicotinic receptor antagonist” is meant a compound which interacts reversibly with nicotinic receptors at, or close to, the agonist binding site, stabilizing the receptor and preventing access for agonists. Nicotinic agonists and competitive antagonists compete for nicotinic binding sites. As inhibition by competitive nicotine antagonists is surmountable by increasing nicotine agonist concentrations, hence the use of the term “competitive.”

By “non-competitive nicotinic antagonists” is meant compounds, which interact with sites distinct from the agonist binding site, and therefore, do not compete with the agonists for binding. The action of non-competitive nicotinic antagonists is not surmountable by nicotinic agonists. Mecamylamine is an example of a non-competitive nicotinic antagonist.

Therefore, the term “nicotinic receptor agonist or antagonist” as used throughout, refers to compounds, which modulate the neuronal nicotinic cholinergic receptor and includes nicotinic acetylcholine receptor subunit compositions. “Nicotinic receptor agonists” are compounds which have nicotinic pharmacology or act on the nicotinic receptor channels. “Nicotinic antagonists” block or compete for the same receptor as nicotine and block ganglia which nicotine stimulates or may act at sites distinct from the nicotinic binding site. This definition includes nicotine receptor partial agonists or agonist/antagonists which can include compounds with affinity for some specific nicotine receptor subunits but no affinity or antagonism at other nicotine receptor subtypes. For example, one category of nicotinic receptor agonists particularly useful in the subject invention are those agonists and partial agonists having affinity and selectivity for the alpha 7 subtype of nicotinic receptor. Depending on the nicotine receptor subunits involved, partial agonists may have reduced side effects and enhanced efficacy. This definition also includes pharmaceutically acceptable salts of, prodrugs of and pharmaceutically acceptable salts of said prodrugs (Sharples, C., Neuronal Nicotinic Receptors, 19 Tocris Reviews 1, (2001)).

Nicotine receptor antagonists or agonists are a large and growing category. A truly exhaustive list of such compounds is not provided herein. It is to be understood that the following discussion is not intended to be exhaustive but to teach how to identify compounds which are encompassed by these terms. The “nicotinic receptor antagonists” useful herein include, but are not limited to, mecamylamine, amantadine, di-hydro-beta-erythroidine (described in Clark and Reuben, 117 Br. J. Pharmacol, 595-606; (1996)), hexamethonium, erysodine, pempidine (described in Banerjee et al., 40 Biochemical Pharmacology, 2105-2110; (1990)), methyllycaconitine, chlorisondamine, trimethaphan, normecamylamine, N-(1,2,2)trimethyl-1-bicyclo[2,2,1,]-heptylbenzenamine, dimethylaminoisocamphane, exoaminonorbornane, 2,2,6,6-tetramethylpiperidine, and 2,2,6,6-tetramethyl-4-aminopiperidine. These references and their test methods are hereby incorporated herein by reference. Additional examples of “nicotine receptor antagonists” include erysodine (Decker, 280 European Journal of Pharmacology, 79-89; (1995)); phenyltropane carboxylic acid methyl esters (Lerner-Marmarosh et al., Life Sciences, 56(3): PL 67-70; (1995)); arylpempidine analogues (Wang et al., 60 Life Sciences, 1271-1277; (1997)); and ibogaine (Daly, 40 (9) Biochemical Pharmacology, 2105-10; (1990)).

The “nicotine receptor agonists” useful herein include, but are not limited to, varenicline, gamma nicotine compounds described in U.S. Pat. Nos. 5,242,934, 5,223,497, and 5,278,045; alpha nicotine compounds described in U.S. Pat. No. 5,232,933; fluorine containing derivatives of nicotine described in U.S. Pat. No. 4,965,074; nicotine itself or N-lower alkyl analogs described in U.S. Pat. No. 5,278,176; nicotine compounds in the (R)-(+)-form described in U.S. Pat. No. 5,227,391; and pyridyalkylpiperidine or pyridylalkylpyrrolidine compounds described in U.S. Pat. No. 5,214,060. Each of the preceding U.S. patents in this paragraph is incorporated by reference herein in its entirety.

It will be recognized by those skilled in the art in light of this disclosure that other nicotinic receptor agonists and antagonists are also useful in the combinations, pharmaceutical compositions, methods and kits of this invention.

Other compounds which may reasonably be expected to be active in this use are disclosed in U.S. Pat. No. 4,837,218 (Alkylated Bicycloalkaneamines for Neurotoxic Injury), U.S. Pat. No. 2,894,987 (N-allyl-2-aminoisocamphane), U.S. Pat. No. 3,148,118 (Analeptically Active Agents), and U.S. Pat. No. 3,164,601 (Analeptically Active N-Substituted Aminonorcamphane Derivatives). These patents are incorporated in their entireties herein by reference.

The nicotinic receptor agonists and antagonists disclosed herein are prepared by methods well known to those skilled in the art. Specifically, the aforementioned patent and patent applications, each of which is incorporated herein by reference, exemplify nicotinic receptor agonists or antagonists which can be used in the combinations, pharmaceutical compositions, methods and kits of this invention, and refer to methods of preparing those nicotinic receptor agonists or antagonists.

Other nicotinic receptor agonists that can be used in the present invention are those compounds described in U.S. Pat. No. 6,410,550 and U.S. Pat. No. 6,605,610, the entire contents of which patents are incorporated herein.

Such nicotinic receptor agonists include compounds of the formula 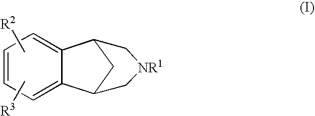
R¹ is hydrogen, (C₁-C₆)alkyl, unconjugated (C₃-C₆)alkenyl, benzyl, XC(═O)R¹³ or —CH₂CH₂—O—(C₁-C₄)alkyl;
R² and R³ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁₋C₆)alkylamino-, [(C₁-C₆)alkyl]₂amino-, —CO₂R⁴, —CONR⁵R⁶, —SO₂NR⁷R⁸, —C(═O)R¹³, —XC(═O)R¹³, aryl-(C₀-C₃)alkyl- or aryl-(C₀-C₃)alkyl-O—, wherein said aryl is selected from phenyl and naphthyl, heteroaryl-(C₀-C₃)alkyl- or heteroaryl-(C₀-C₃)alkyl-O—, wherein said heteroaryl is selected from five to seven membered aromatic rings containing from one to four heteroatoms selected from oxygen, nitrogen and sulfur; X²(C₀-C₆)alkyl- and X²(C₁-C₆)alkoxy-(C₀-C₆)alkyl-, wherein X² is absent or X² is (C₁-C₆)alkylamino- or [(C₁-C₆)alkyl]₂amino-, and wherein the (C₀-C₆)alkyl- or (C₁-C₆)alkoxy-(C₀-C₆)alkyl- moieties of said X²(C₀-C₆)alkyl- or X²(C₁-C₆)alkoxy-(C₀-C₆)alkyl- contains at least one carbon atom, and wherein from one to three of the carbon atoms of said (C₀-C₆)alkyl- or (C₁-C₆)alkoxy-(C₀-C₆)alkyl- moieties may optionally be replaced by an oxygen, nitrogen or sulfur atom, with the proviso that any two such heteroatoms must be separated by at least two carbon atoms, and wherein any of the alkyl moieties of said (C₀-C₆)alkyl- or (C₁₋C₆)alkoxy-(C₀-C₆)alkyl- groups may be optionally substituted with from two to seven fluorine atoms, and wherein one of the carbon atoms of each of the alky moieties of said aryl-(C₀-C₃)alkyl- and said heteroaryl-(C₀-C₃)alkyl- may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluorine atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)alkylamino-, [(C₁-C₆)alkyl]₂amino-, —CO₂R⁴, —CONR⁵R⁶, —SO₂NR⁷R⁸, —C(═O)R¹³ and —XC(═O)R¹³;
or R² and R³, together with the carbons to which they are attached, form a four to seven membered monocyclic, or a ten to fourteen membered bicyclic, carbocyclic ring that can be saturated or unsaturated, wherein from one to three of the non-fused carbon atoms of said monocyclic rings, and from one to five of the carbon atoms of said bicyclic rings that are not part of the benzo ring shown in formula I, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkyl- or (C₁-C₆)alkoxy-(C₀-C₆)alkyl-, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino-, [(C₁-C₆)alkyl]₂amino-, —CO₂R⁴, —CONR⁵R⁶, —SO₂NR⁷R⁸, —C(═O)R¹³, and —XC(═O)R¹³;
each R⁴, R⁵, R⁶, R⁷, R⁸ and R¹³ is selected, independently, from hydrogen and (C₁-C₆) alkyl, or R⁵ and R⁶, or R⁷ and R⁸ together with the nitrogen to which they are attached, form a pyrrolidine, piperidine, morpholine, azetidine, piperazine, —N-(C₁-C₆)alkylpiperazine or thiomorpholine ring, or a thiomorpholine ring wherein the ring sulfur is replaced with a sulfoxide or sulfone; and
each X is, independently, (C₁-C₆)alkylene;
with the proviso that: (a) at least one of R¹, R² and R³ must be the other than hydrogen, and (b) when R² and R³ are hydrogen, R¹ cannot be hydrogen, (C₁-C₆)alkyl, or unconjugated (C₃-C₆)alkenyl, and pharmaceutically acceptable salts of such compounds.

Examples of specific compounds of the formula I are the following compounds, which, in the instances where there is a center or centers of asymmetry in the molecule, may comprise a racemic mixture or the single enantiomer:
5,13-diazatetracyclo[9.3.1.0^{2,10}.0^{4,8}]pentadeca-2,4(8),9-trien-6-one;
6-oxo-5-oxa-7,13-diazatetracyclo[9.3.1.0^{2,10}.0^{4,8}]pentadeca-2(10),3,6,8-tetraene;
2-fluoro-N-(4-hydroxy-10-aza-tricyclo[6.3.1.0^{2,7}]dodeca-2(7),3,5-trien-5-yl)benzamide;
6-methyl-5-thia-7,13-diazatetracyclo[9.3.1.0^{2,10}.0^{4,8}]pentadeca-2(10),3,6,8-tetraene;
6-methyl-7-propyl-5,7,13-triazatetracyclo[9.3.1.0^{2,10}.0^{4,8}]pentadeca-2(10),3,5,8-tetraene;
5,7,13-triazatetracyclo[9.3.1.0^{2,10}.0^{4,8}]pentadeca-2(10),3,5,8-tetraene;
7-methyl-5,7,13-triazatetracyclo[9.3.1.0^{2,10}.0^{4,8}]pentadeca-2(10),3,5,8-tetraene;
6-methyl-5,7,13-triazatetracyclo[9.3.1.0^{2,10}.0^{4,8}]pentadeca-2(10),3,5,8-tetraene;
6,7-dimethyl-5,7,13-triazatetracyclo[9.3.1.0^{2,10}.0^{4,8}]pentadeca-2(10),3,5,8-tetraene;
7-propyl-5,7,13-triazatetracyclo[9.3.1.0^{2,10}.0^{4,8}]pentadeca-2(10),3,5,8-tetraene;
7-butyl-5,7,13-triazatetracyclo[9.3.1.0^{2,10}.0^{4,8}]pentadeca-2(10),3,5,8-tetraene;
6-methyl-7-isobutyl-5,7,13-triazatetracyclo[9.3.1.0^{2,10}.0^{4,8}]pentadeca-2(10),3,5,8-tetraene;
7-phenyl-5,7,13-triazatetracyclo[9.3.1.0^{2,10}.0^{4,8}]pentadeca-2(10),3,5,8-tetraene;
6-methyl-7-phenyl-5,7,13-triazatetracyclo[9.3.1.0^{2,10}.0^{4,8}]pentadeca-2(10),3,5,8-tetraene;
7-neopentyl-5,7,123-triazatetracyclo[9.3.1.0^{2,10}.0^{4,8}]pentadeca-2(10),3,5,8-tetraene;
6-methyl-7-neopentyl-5,7,13-triazatetracyclo[9.3.1.0^{2,10}.0^{4,8}]pentadeca-2 (10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
14-methyl-5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
7-methyl-5-oxa-6,13-diazatetracyclo[9.3.1.0^{2,10}.0^{4,8}]pentadeca-2,4(8),6,9-tetraene;
4-methyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
4-amino-10-azatricyclo[6.3.1.0^{2,7}]dodeca-2(7),3,5-triene;
N¹-[10-azatricyclo[6.3.1.0^{2,7}]dodeca-2(7),3,5-trien-4-yl]acetamide;
4,5-dinitro-10-aza-tricyclo[6.3.1.0^{2,7}]dodeca-2(7),3,5-triene;
4,5-difluoro-10-aza-tricyclo[6.3.1.0^{2,7}]dodeca-2(7),3,5-triene;
4-chloro-10-azatricyclo[6.3.1.0^{2,7}]dodeca-2(7),3,5-triene;
3-(10-azatricyclo[6.3.1.0^{2,7}]dodeca-2(7),3,5-trien-4-yl)-5-methyl-1,2,4-oxadiazole;
10-azatricyclo[6.3.1.0^{2,7}]dodeca-2(7),3,5-trien-4-ol;
4,5-dichloro-10-azatricyclo[6.3.1.0^{2,7}]dodeca-2(7),3,5-triene;
N⁴,N⁴-dimethyl-10-azatricyclo[6.3.1.0^{2,7}]dodeca-2(7),3,5-triene-4-sulfonamide;
4-(1-pyrrolidinylsulfonyl)-10-azatricyclo[6.3.1.0^{2,7}]dodeca-2(7),3,5-triene;
1-(10-azatricyclo[6.3.1.0^{2,7}]dodeca-2(7),3,5-trien-4-yl)-1-ethanone;
3-trifluoromethyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
3-fluoro-10-aza-tricyclo[6.3.1.0^{2,7}]dodeca-2(7),3,5-triene;
10-azatricyclo[6.3.1.0^{2,7}]dodeca-2(7),3,5-trien-4-yl cyanide;
4-fluoro-10-aza-tricyclo[6.3.1.0^{2,7}]dodeca-2(7),3,5-triene;
5,14-diazatetracyclo[10.3.1.0^{2,11}.0^{4,9}]hexadeca-2(11),3,5,7,9-pentaene;
6-methyl-5,14-diazatetracyclo[10.3.1.0^{2,11}.0^{4,9}]hexadeca-2(11),3,5,7,9-pentaene;
7-methyl-5,14-diazatetracyclo[10.3.1.0^{2,11}.0^{4,9}]hexadeca-2(11),3,5,7,9-pentaene;
7-ethyl-5,14-diazatetracyclo[10.3.1.0^{2,11}.0^{4,9}]hexadeca-2(11),3,5,7,9-pentaene;
8-methyl-5,14-diazatetracyclo[10.3.1.0^{2,11}.0^{4,9}]hexadeca-2(11),3,5,7,9-pentaene;
5,14-diazatetracyclo[10.3.1.0^{2,11}.0^{4,9}]hexadeca-2(11),3,7,9-tetraen-6-one;
6-chloro-5,14-diazatetracyclo[10.3.1.0^{2,11}.0^{4,9}]hexadeca-2(11),3,5,7,9-pentaene;
6-methoxy-5,14-diazatetracyclo[10.3.1.0^{2,11}.0^{4,9}]hexadeca-2(11),3,5,7,9-pentaene;
6-chloro-10-fluoro-5,14-diazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,7,9-tetraen-6-one;
6-chloro-3-fluoro-5,14-diazatetracyclo[10.3.1.0^{2,11}.0^{4,9}]hexadeca-2(11),3,5,7,9-pentaene;
and pharmaceutically acceptable salts thereof.

Other embodiments of compounds of formula I that can be used in the subject invention are:
6-methyl-5,7-dioxo-6,13-diazatetracyclo[9.3.1.0^{2,10}.0^{4,8}])pentadeca-2(10),3,8-triene;
6-methyl-5-oxo-6,13-diazatetracyclo[9.3.1.0^{2,10}.0^{4,8}]pentadeca-2(10),3,8-triene;
5,7-dimethyl-6-oxo-5,7,13-triazatetracyclo[9.3.1.0^{2,10}.0^{4,8}]pentadeca-2(10),3,8-triene;
5,7-dioxo-6,13-diazatetracyclo[9.3.1.0^{2,10}.0^{4,8}]pentadeca-2(10),3,8-triene;
5-oxo-6,13-diazatetracyclo[9.3.1.0^{2,10}.0^{4,8}]pentadeca-2(10),3,8-triene;
6-oxo-5,7,13-triazatetracyclo[9.3.1.0^{2,10}.0^{4,8}]pentadeca-2(10),3,8-triene;
6-methyl-5-thia-5-dioxo-6,13-diazatetracyclo[9.3.1.0^{2,10}.0^{4,8}]pentadeca-2(10),3,6,8-tetraene;
7-dimethylamino-5-thia-5-dioxo-6,13-diazatetracyclo[9.3.1.0^{2,10}.0^{4,8}]pentadeca-2(10),3,6,8-tetraene;
6,7-dioxo-5,8,14-triazatetracyclo[10.3.1.0^{2,11}.0^{4,9}]hexadeca-2(11),3,9-triene;
5,8-dimethyl-6,7-dioxo-5,8,14-triazatetracyclo[10.3.1.0^{2,11}.0^{4,9}]hexadeca-2(11),3,9-triene;
5-oxa-7-methyl-6-oxo-7,13-diazatetracyclo[9.3.1.0^{2,10}.0^{4,8}]pentadeca-2(10),3,8-triene;
5-fluoro-10-aza-tricyclo[6.3.1.0^{2,7}]dodeca-2(7),3,5-triene-4-carbonitrile;
4-ethynyl-5-fluoro-10-aza-tricyclo[6.3.1.0^{2,7}]dodeca-2(7),3,5-triene;
5-ethynyl-10-aza-tricyclo[6.3.1.0^{2,7}]dodeca-2(7),3,5-triene-4-carbonitrile;
5-chloro-10-aza-tricyclo[6.3.1.0^{2,7}]dodeca-2(7),3,5-triene-4-carbonitrile;
4-ethynyl-5-chloro-10-aza-tricyclo[6.3.1.0^{2,7}]dodeca-2(7),3,5-triene;
4-fluoro-5-trifluoromethyl-10-aza-tricyclo[6.3.1.0^{2,7}]dodeca-2(7),3,5-triene;
4-chloro-5-trifluoromethyl-10-aza-tricyclo[6.3.1.0^{2,7}]dodeca-2(7),3,5-triene;
5-trifluoromethyl-10-aza-tricyclo[6.3.1.0^{2,7}]dodeca-2(7),3,5-triene-4-carbonitrile;
4-ethynyl-5-trifluoromethyl-10-aza-tricyclo[6.3.1.0^{2,7}]dodeca-2(7),3,5-triene;
4,5-bistrifluoromethyl-10-aza-tricyclo[6.3.1.0^{2,7}]dodeca-2(7),3,5-triene;
and pharmaceutically acceptable salts thereof.

Particularly, preferred enantiomers of the compounds of formula I for use in the subject invention are:
(+)-5,13-diazatetracyclo[9.3.1.0^{2,10}.0^{4,8}]pentadeca-2,4(8),9-trien-6-one;
(+)-6-oxo-5-oxa-7,13-diazatetracyclo[9.3.1.0^{2,10}.0^{4,8}]pentadeca-2(10),3,6,8-tetraene;
(+)-2-fluoro-N-(4-hydroxy-10-aza-tricyclo[6.3.1.0^{2,7}]dodeca-2(7),3,5-trien-5-yl)benzamide;
(+)-6-methyl-5-thia-7,13-diazatetracyclo[9.3.1.0^{2,10}.0^{4,8}]pentadeca-2(10),3,6,8-tetraene;
(+)-6-methyl-7-propyl-5,7,13-triazatetracyclo[9.3.1.0^{2,10}.0^{4,8}]pentadeca-2(10),3,5,8-tetraene;
(+)-7-methyl-5,7,13-triazatetracyclo[9.3.1.0^{2,10}.0^{4,8}]pentadeca-2(10),3,5,8-tetraene;
(+)-6,7-dimethyl-5,7,13-triazatetracyclo[9.3.1.0^{2,10}.0^{4,8}]pentadeca-2(10),3,5,8-tetraene;
(+)-7-propyl-5,7,13-triazatetracyclo[9.3.1.0^{2,10}.0^{4,8}]pentadeca-2(10),3,5,8-tetraene;
(+)-7-butyl-5,7,13-triazatetracyclo[9.3.1.0^{2,10}.0^{4,8}]pentadeca-2(10),3,5,8-tetraene;
(+)-6-methyl-7-isobutyl-5,7,13-triazatetracyclo[9.3.1.0^{2,10}.0^{4,8}]pentadeca-2(10),3,5,8-tetraene;
(+)-7-phenyl-5,7,13-triazatetracyclo[9.3.1.0^{2,10}.0^{4,8}]pentadeca-2(10),3,5,8-tetraene;
(+)-6-methyl-7-phenyl-5,7,13-triazatetracyclo[9.3.1.0^{2,10}.0^{4,8}]pentadeca-2(10),3,5,8-tetraene;
(+)-7-neopentyl-5,7,13-triazatetracyclo[9.3.1.0^{2,10}.0^{4,8}]pentadeca-2(10),3,5,8-tetraene;
(+)-6-methyl-7-neopentyl-5,7,13-triazatetracyclo[9.3.1.0^{2,10}.0^{4,8}]pentadeca-2(10),3,5,8-tetraene;
(+)-5-oxa-7,13-diazatetracyclo[9.3.1.0^{2,10}.0^{4,8}]pentadeca-2(10),3,6,8-tetraene;
(+)-6-methyl-5-oxa-7,13-diazatetracyclo[9.3.1.0^{2,10}.0^{4,8}]pentadeca-2(10),3,6,8-tetraene;
(+)-7-methyl-5-oxa-6,13-diazatetracyclo[9.3.1.0^{2,10}.0^{4,8}]pentadeca-2,4(8),6,9-tetraene;
(+)-4-methyl-10-aza-tricyclo[6.3.1.0^{2,7}]dodeca-2 (7),3,5-triene;
(+)-4-nitro-10-azatricyclo[6.3.1.0^{2,7}]dodeca-2(7),3,5-triene;
(+)-4-amino-10-azatricyclo[6.3.1.0^{2,7}]dodeca-2(7),3,5-triene;
(+)-N¹-[10-azatricyclo[6.3.1.0^{2,7}]dodeca-2(7),3,5-trien-4-yl]acetamide;
(+)-4-chloro-10-azatricyclo[6.3.1.0^{2,7}]dodeca-2(7),3,5-triene;
(+)-3-(10-azatricyclo[6.3.1.0^{2,7}]dodeca-2(7),3,5-trien-4-yl)-5-methyl-1,2,4-oxadiazole;
(+)-10-azatricyclo[6.3.1.0^{2,7}]dodeca-2(7),3,5-trien-4-ol;
(+)-N⁴,N⁴-dimethyl-10-azatricyclo[6.3.1.0^{2,7}]dodeca-2(7),3,5-triene-4-sulfonamide;
(+)-4-(1-pyrrolidinylsulfonyl)-10-azatricyclo[6.3.1.0^{2,7}]dodeca-2(7),3,5-triene;
(+)-1-(10-azatricyclo[6.3.1.0^{2,7}]dodeca-2(7),3,5-trien-4-yl)-1-ethanone;
(+)-3-trifluoromethyl-10-aza-tricyclo[6.3.1.0^{2,7}]dodeca-2(7),3,5-triene;
(+)-4-trifluoromethyl-10-aza-tricyclo[6.3.1.0^{2,7}]dodeca-2(7),3,5-triene;
(+)-3-fluoro-10-aza-tricyclo[6.3.1.0^{2,7}]dodeca-2(7),3,5-triene;
(+)-10-azatricyclo[6.3.1.0^{2,7}]dodeca-2(7),3,5-trien-4-yl cyanide;
(+)-4-fluoro-10-aza-tricyclo[6.3.1.0^{2,7}]dodeca-2(7),3,5-triene;
(+)-6-methyl-5-oxo-6,13-diazatetracyclo[9.3.1.0²¹⁰.0^{4,8}]pentadeca-2(10),3,8-triene;
(+)-5-oxo-6,13-diazatetracyclo[9.3.1.0^{2,10}.0^{4,8}]pentadeca-2(10),3,8-triene;
(+)-6-methyl-5-thia-5-dioxo-6,13-diazatetracyclo[9.3.1.0^{2,10}.0^{4,8}]pentadeca-2(10),3,6,8-tetraene;
(+)-7-dimethylamino-5-thia-5-dioxo-6,13-diazatetracyclo[9.3.1.0^{2,10}.0^{4,8}]pentadeca-2(10),3,6,8-tetraene;
(+)-5-oxa-7-methyl-6-oxo-7,13-diazatetracyclo[9.3.1.0^{2,10}.0^{4,8}]pentadeca-2(10),3,8-triene;
(+)-5-fluoro-10-aza-tricyclo[6.3.1.0^{2,7}]dodeca-2(7),3,5-triene-4-carbonitrile;
(+)-4-ethynyl-5-fluoro-10-aza-tricyclo[6.3.1.0^{2,7}]dodeca-2(7),3,5-triene;
(+)-5-ethynyl-10-aza-tricyclo[6.3.1.0^{2,7}]dodeca-2(7),3,5-triene-4-carbonitrile;
(+)-5-chloro-10-aza-tricyclo[6.3.1.0^{2,7}]dodeca-2(7),3,5-triene-4-carbonitrile;
(+)-4-ethynyl-5-chloro-10-aza-tricyclo[6.3.1.0^{2,7}]dodeca-2(7),3,5-triene;
(+)-4-fluoro-5-trifluoromethyl-10-aza-tricyclo[6.3.1.0^{2,7}]dodeca-2(7),3,5-triene;
(+)-4-chloro-5-trifluoromethyl-10-aza-tricyclo[6.3.1.0^{2,7}]dodeca-2(7),3,5-triene;
(+)-5-trifluoromethyl-10-aza-tricyclo[6.3.1.0^{2,7}]dodeca-2(7),3,5-triene-4-carbonitrile;
(+)-4-ethynyl-5-trifluoromethyl-10-aza-tricyclo[6.3.1.0^{2,7}]dodeca-2(7),3,5-triene;
(+)-5,14-diazatetracyclo[10.3.1.0^{2,11}.0^{4,9}]hexadeca-2(11),3,5,7,9-pentaene;
(+)-6-methyl-5,14-diazatetracyclo[10.3.1.0^{2,11}.0^{4,9}]hexadeca-2(11),3,5,7,9-pentaene;
(+)-7-methyl-5,14-diazatetracyclo[10.3.1.0^{2,11}.0^{4,9}]hexadeca-2(11),3,5,7,9-pentaene;
(+)-7-ethyl-5,14-diazatetracyclo[10.3.1.0^{2,11}.0^{4,9}]hexadeca-2(11),3,5,7,9-pentaene;
(+)-8-methyl-5,14-diazatetracyclo[10.3.1.0^{2,11}.0^{4,9}]hexadeca-2(11),3,5,7,9-pentaene;
(+)-5,14-diazatetracyclo[10.3.1.0^{2,11}.0^{4,9}]hexadeca-2(11),3,7,9-tetraen-6-one;
(+)-6-chloro-5,14-diazatetracyclo[10.3.1.0^{2,11}.0^{4,9}]hexadeca-2(11),3,5,7,9-pentaene;
(+)-6-methoxy-5,14-diazatetracyclo[10.3.1.0^{2,11}.0^{4,9}]hexadeca-2(11),3,5,7,9-pentaene;
(+)-6-chloro-10-fluoro-5,14-diazatetracyclo[10.3.1.0^{2,11}.0^{4,9}]hexadeca-2(11),3,5,7,9-pentaene;
(+)-5,8,14-triazatetracyclo[10.3.1.0^{2,11}.0^{4,9}]hexadeca-2(11),3,7,9-tetraen-6-one;
(+)-6-chloro-3-fluoro-5,14-diazatetracyclo[10.3.1.0^{2,11}.0^{4,9}]hexadeca-2(11),3,5,7,9-pentaene;
and pharmaceutically acceptable salts thereof.

In addition, other enantiomers of the compounds of formula I that are preferred for use in the present invention are:
(−)-5,13-diazatetracyclo[9.3.1.0^{2,10}.0^{4,8}]pentadeca-2,4(8),9-trien-6-one;
(−)-6-oxo-5-oxa-7,13-diazatetracyclo[9.3.1.0^{2,10}.0^{4,8}]pentadeca-2(10),3,6,8-tetraene;
(−)-2-fluoro-N-(4-hydroxy-10-aza-tricyclo[6.3.1.0^{2,7}]dodeca-2(7),3,5-trien-5-yl)benzamide;
(−)-6-methyl-5-thia-7,13-diazatetracyclo[9.3.1.0^{2,10}.0^{4,8}]pentadeca-2(10),3,6,8-tetraene;
(−)-6-methyl-7-propyl-5,7,13-triazatetracyclo[9.3.1.0^{2,10}.0^{4,8}]pentadeca-2(10),3,5,8-tetraene;
(−)-7-methyl-5,7,13-triazatetracyclo[9.3.1.0^{2,10}.0^{4,8}]pentadeca-2(10),3,5,8-tetraene;
(−)-6,7-dimethyl-5,7,13-triazatetracyclo[9.3.1.0^{2,10}.0^{4,8}]pentadeca-2(10),3,5,8-tetraene;
(−)-7-propyl-5,7,13-triazatetracyclo[9.3.1.0^{2,10}.0^{4,8}]pentadeca-2(10),3,5,8-tetraene;
(−)-7-butyl-5,7,13-triazatetracyclo[9.3.1.0^{2,10}.0^{4,8}]pentadeca-2(10),3,5,8-tetraene;
(−)-6-methyl-7-isobutyl-5,7,13-triazatetracyclo[9.3.1.0^{2,10}.0^{4,8}]pentadeca-2(10),3,5,8-tetraene;
(−)-7-phenyl-5,7,13-triazatetracyclo[9.3.1.0^{2,10}.0^{4,8}]pentadeca-2(10),3,5,8-tetraene;
(−)-6-methyl-7-phenyl-5,7,13-triazatetracyclo[9.3.1.0^{2,10}.0^{4,8}]pentadeca-2(10),3,5,8-tetraene;
(−)-7-neopentyl-5,7,13-triazatetracyclo[9.3.1.0^{2,10}.0^{4,8}]pentadeca-2(10),3,5,8-tetraene;
(−)-6-methyl-7-neopentyl-5,7,13-triazatetracyclo[9.3.1.0^{2,10}.0^{4,8}]pentadeca-2(10),3,5,8-tetraene;
(−)-5-oxa-7,13-diazatetracyclo[9.3.1.0^{2,10}.0^{4,8}]pentadeca-2(10),3,6,8-tetraene;
(−)-6-methyl-5-oxa-7,13-diazatetracyclo[9.3.1.0^{2,10}.0^{4,8}]pentadeca-2(10),3,6,8-tetraene;
(−)-7-methyl-5-oxa-6,13-diazatetracyclo[9.3.1.0^{2,10}.0^{4,8}])pentadeca-2,4(8),6,9-tetraene;
(−)-4-methyl-10-aza-tricyclo[6.3.1.0^{2,7}]dodeca-2(7),3,5-triene;
(−)-4-nitro-10-azatricyclo[6.3.1.0^{2,7}]dodeca-2(7),3,5-triene;
(−)-4-amino-10-azatricyclo[6.3.1.0^{2,7}]dodeca-2(7),3,5-triene;
(−)-N¹-[10-azatricyclo[6.3.1.0^{2,7}]dodeca-2(7),3,5-trien-4-yl]acetamide;
(−)-4-chloro-10-azatricyclo[6.3.1.0^{2,7}]dodeca-2(7),3,5-triene;
(−)-3-(10-azatricyclo[6.3.1.0^{2,7}]dodeca-2(7),3,5-trien-4-yl)-5-methyl-1,2,4-oxadiazole;
(−)-10-azatricyclo[6.3.1.0^{2,7}]dodeca-2(7),3,5-trien-4-ol;
(−)-N⁴,N⁴-dimethyl-10-azatricyclo[6.3.1.0^{2,7}]dodeca-2(7),3,5-triene-4-sulfonamide;
(−)-4-(1-pyrrolidinylsulfonyl)-10-azatricyclo[6.3.1.0^{2,7}]dodeca-2(7),3,5-triene;
(−)-1-(10-azatricyclo[6.3.1.0^{2,7}]dodeca-2(7),3,5-trien-4-yl)-1-ethanone;
(−)-3-trifluoromethyl-10-aza-tricyclo[6.3.1.0^{2,7}]dodeca-2(7),3,5-triene;
(−)-4-trifluoromethyl-10-aza-tricyclo[6.3.1.0^{2,7}]dodeca-2(7),3,5-triene;
(−)-3-fluoro-10-aza-tricyclo[6.3.1.0^{2,7}]dodeca-2(7),3,5-triene;
(−)-10-azatricyclo[6.3.1.0^{2,7}]dodeca-2(7),3,5-trien-4-yl cyanide;
(−)-4-fluoro-10-aza-tricyclo[6.3.1.0^{2,7}]dodeca-2(7),3,5-triene;
(−)-6-methyl-5-oxo-6,13-diazatetracyclo[9.3.1.0^{2,10}.0^{4,8}]pentadeca-2(10),3,8-triene;
(−)-5-oxo-6,13-diazatetracyclo[9.3.1.0^{2,10}.0^{4,8}]pentadeca-2(10),3,8-triene;
(−)-6-methyl-5-thia-5-dioxo-6,13-diazatetracyclo[9.3.1.0^{2,10}.0^{4,8}]pentadeca-2(10),3,6,8-tetraene;
(−)-7-dimethylamino-5-thia-5-dioxo-6,13-diazatetracyclo[9.3.1.0^{2,10}.0^{4,8}]pentadeca-2(10),3,6,8-tetraene;
(−)-5-oxa-7-methyl-6-oxo-7,13-diazatetracyclo[9.3.1.0^{2,10}.0^{4,8}]pentadeca-2(10),3,8-triene;
(−)-5-fluoro-10-aza-tricyclo[6.3.1.0^{2,7}]dodeca-2(7),3,5-triene-4-carbon itrile;
(−)-4-ethynyl-5-fluoro-10-aza-tricyclo[6.3.1.0^{2,7}]dodeca-2(7),3,5-triene;
(−)-5-ethynyl-10-aza-tricyclo[6.3.1.0^{2,7}]dodeca-2(7),3,5-triene-4-carbonitrile;
(−)-5-chloro-10-aza-tricyclo[6.3.1.0^{2,7}]dodeca-2(7),3,5-triene-4-carbon itrile;
(−)-4-ethynyl-5-chloro-10-aza-tricyclo[6.3.1.0^{2,7}]dodeca-2(7),3,5-triene;
(−)-4-fluoro-5-trifluoromethyl-10-aza-tricyclo[6.3.1.0^{2,7}]dodeca-2(7),3,5-triene;
(−)-4-chloro-5-trifluoromethyl-10-aza-tricyclo[6.3.1.0^{2,7}]dodeca-2(7),3,5-triene;
(−)-5-trifluoromethyl-10-aza-tricyclo[6.3.1.0^{2,7}]dodeca-2(7),3,5-triene-4-carbonitrile;
(−)-4-ethynyl-5-trifluoromethyl-10-aza-tricyclo[6.3.1.0^{2,7}]dodeca-2(7),3,5-triene;
(−)-5,14-diazatetracyclo[10.3.1.0^{2,11}.0^{4,9}]hexadeca-2(11),3,5,7,9-pentaene;
(−)-6-methyl-5,14-diazatetracyclo[10.3.1.0^{2,11}.0^{4,9}]hexadeca-2(11),3,5,7,9-pentaene;
(−)-7-methyl-5,14-diazatetracyclo[10.3.1.0^{2,11}.0^{4,9}]hexadeca-2(11),3,5,7,9-pentaene;
(−)-7-ethyl-5,14-diazatetracyclo[10.3.1.0^{2,11}.0^{4,9}]hexadeca-2(11),3,5,7,9-pentaene;
(−)-8-methyl-5,14-diazatetracyclo[10.3.1.0^{2,11}.0^{4,9}]hexadeca-2(11),3,5,7,9-pentaene;
(−)-5,14-diazatetracyclo[10.3.1.0^{2,11}.0^{4,9}]hexadeca-2(11),3,7,9-tetraen-6-one;
(−)-6-chloro-5,14-diazatetracyclo[10.3.1.0^{2,11}.0^{4,9}]hexadeca-2(11),3,5,7,9-pentaene;
(−)-6-methoxy-5,14-diazatetracyclo[10.3.1.0^{2,11}.0^{4,9}]hexadeca-2(11),3,5,7,9-pentaene;
(−)-6-chloro-10-fluoro-5,14-diazatetracyclo[10.3.1.0^{2,11}.0^{4,9}]hexadeca-2(11),3,5,7,9-pentaene;
(−)-5,8,14-triazatetracyclo[10.3.1.0^{2,11}.0^{4,9}]hexadeca-2(11),3,7,9-tetraen-6-one;
(−)-6-chloro-3-fluoro-5,14-diazatetracyclo[10.3.1.0^{2,11}.0^{4,9}]hexadeca-2(11),3,5,7,9-pentaene;
and pharmaceutically acceptable salts thereof.

Synthesis of compounds of formula I is described in, for example, U.S. Pat. No. 6,410,550 which is incorporated by reference herein in its entirety.

Other nicotinic receptor agonists that are useful in the present invention are the compounds described in U.S. Pat. No. 6,809,094; U.S. Ser. No. 10/163,564, filed Jun. 6, 2002 (U.S. 2003/0045540A1, published Mar. 6, 2003); and U.S. Ser. No. 10/262,257, filed Oct. 1, 2002 (U.S. 2003/0153595A1, published Aug. 14, 2003). The entire contents of the aforementioned U.S. patents and patent applications are incorporated herein.

For example, U.S. Pat. No. 6,809,094 described compounds of the formula II 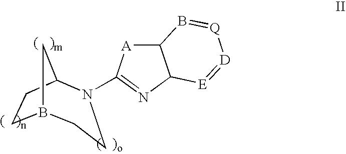
wherein
n=1-2;
m=1-2;
o=1-2;
A=O, S or NR¹;
B=N or CR²;
Q=N or CR³;
D=N or CR⁴;
E=N or CR⁵;
R¹ is H, a straight chain or branched (C₁-C₈)alkyl, C(═O)OR⁶, CH₂R⁶, C(═O)NR⁶R⁷, C(═O)R⁶, or SO₂R⁶;
each R², R³, R⁴ and R⁵ is independently selected from F, Cl, Br, I, nitro, cyano, CF₃, —NR⁶R⁷, —NR⁶C(═O)R⁷, —NR⁶C(═O)NR⁷R⁸, —NR⁶C(═O)OR⁷, —NR⁶S(═O)₂R⁷, —NR⁶S(═O)₂NR⁷R⁸, —OR⁶, —OC(═O)R⁶, —OC(═O)OR⁶, —OC(═O)NR⁶R⁷, —OC(═O)SR⁶, —C(═O)OR⁶, —C(═O)R⁶, —C(═O)NR⁶R⁷, —SR⁶, —S(═O)R⁶, —S(═O)₂R⁶, —S(═O)₂NR⁶R⁷, and R⁶;
each R⁶, R⁷, and R⁸ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, 5-11 membered heterobicycloalkenyl, (C₆-C₁₁)aryl, and 5-12 membered heteroaryl; wherein each R⁶, R⁷, and R⁸ is optionally substituted with from one to six substituents, independently selected from F, Cl, Br, I, nitro, cyano, CF₃, —NR⁹R¹⁰, —NR⁹C(═O)R¹⁰, —NR⁹C(═O)NR¹⁰R¹¹, —NR⁹C(═O)OR¹⁰, —NR⁹S(═O)₂R¹⁰, —NR⁹S(═O)₂NR¹⁰R¹¹, —OR⁹, —OC(═O)R⁹, —OC(═O)OR⁹, —OC(═O)NR⁹R¹⁰, —OC(═O)SR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹;
each R⁹, R¹⁰ and R¹¹ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, (5-11 membered) heterobicycloalkenyl, (C₆-C₁₁)aryl or 5-12 membered heteroaryl; wherein each R⁹, R¹⁰ and R¹¹ is optionally substituted with from one to six substituents independently selected from F, Cl, Br, I, nitro, cyano, CF₃, —NR¹²R¹³, —NR¹²C(═O)R¹³, —NR¹²C(═O)NR¹³R¹⁴, —NR¹²C(═O)OR¹³, —NR¹²S(═O)₂R¹³, —NR¹²S(═O)₂NR¹³R¹⁴, —OR¹², —OC(═O)R¹², —OC(═O)OR¹², —OC(═O)NR¹²R¹³, —OC(═O)SR¹², —C(═O)OR¹², —C(═O)R¹², —C(═O)NR¹²R¹³, —SR¹², —S(═O)R¹², —S(═O)₂R¹², —S(═O)₂NR¹²R¹³ and R¹²;
each R¹², R¹³, and R¹⁴ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, 5-11 membered heterobicycloalkenyl, (C₆-C₁₁)aryl and (5-12 membered) heteroaryl;
or R² and R³, or R³ and R⁴, or R⁴ and R⁵, may form another 6-membered aromatic or heteroaromatic ring sharing B and Q, or Q and D, or D and E, respectively, and may be optionally substituted with from one to four substitutuents independently selected from the group of radicals set forth in the definition of R⁶, R⁷ and R⁸ above;
and all enantiomeric, diastereomeric, and tautomeric isomers and pharmaceutically acceptable salts thereof;
which are useful in the combinations of the present invention. Formula II encompasses all enantiomeric, diastereomeric, and tautomeric isomers.

Examples of specific compounds of formula II that are useful in the subject invention are the following compounds and their pharmaceutically acceptable salts:
4-oxazolo[5,4-b]pyridin-2-yl-1,4-diazabicyclo[3.2.2]nonane;
4-oxazolo[5,4-c]pyridin-2-yl-1,4-diazabicyclo[3.2.2]nonane;
4-oxazolo[4,5-c]pyridin-2-yl-1,4-diazabicyclo[3.2.2]nonane;
4-oxazolo[4,5-b]pyridin-2-yl-1,4-diazabicyclo[3.2.2]nonane;
4-(5-methyl-oxazolo[4,5-b]pyridin-2-yl)-1,4-diazabicyclo[3.2.2]nonane;
4-(6-phenyl-oxazolo[5,4-b]pyridin-2-yl)-1,4-diazabicyclo[3.2.2]nonane;
4-(6-bromo-oxazolo[4,5-b]pyridin-2-yl)-1,4-diazabicyclo[3.2.2]nonane; and
4-(6-phenyl-oxazolo[4,5-b]pyridin-2-yl)-1,4-diazabicyclo[3.2.2]nonane.

The synthesis of the compounds of formula II is described in U.S. Pat. No. 6,809,094, the entire content of which is incorporated herein.

U.S. Ser. No. 10/163,564, filed Jun. 6, 2002 (U.S. 2003/0045540A1, published Mar. 6, 2003), the entire content of which is incorporated herein, describes compounds of the following formula III and their synthesis which are nicotinic receptor agonists that are useful in the combinations of the subject invention: 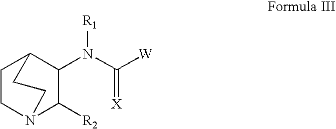
wherein W is 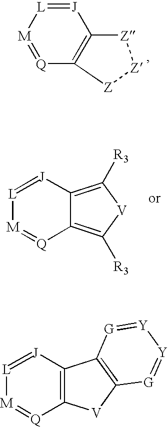
provided that the bond between the —C(═X)— group and the W group may be attached at any available carbon atom within the W group as provided in R₃, R₆, and R₁₅;
X is O, or S;
each R₁ is H, alkyl, cycloalkyl, halogenated alkyl, substituted phenyl, or substituted naphthyl;
R₂ is H, alkyl, halogenated alkyl, substituted alkyl, cycloalkyl, or aryl;
Z---Z′---Z″ is selected from N(R₄)—C(R₃)═C(R₃), N═C(R₃)—C(R₁₅)₂, C(R₃)═C(R₃)—N(R₄), C(R₃)₂—N(R₄)—C(R₃)₂, C(R₁₅)₂—C(R₃)═N, N(R₄)—C(R₃)₂—C(R₃)₂, C(R₃)₂—C(R₃)₂—N(R₄), O—C(R₃)═C(R₃), O—C(R₃)₂—C(R₃)₂, C(R₃)₂—O—C(R₃)₂, C(R₃)═C(R₃)—O, C(R₃)₂—C(R₃)₂—O, S—C(R₃)═C(R₃), S—C(R₃)₂—C(R₃)₂, C(R₃)₂—S—C(R₃)₂, C(R₃)═C(R₃)—S, or C(R₃)₂—C(R₃)₂—S;
each R₃ is independently a bond to the core molecule provided that only one R₃ and no R₆ or R₁₅ is also said bond, H, F, Br, Cl, I, alkyl, substituted alkyl, halogenated alkyl, alkenyl, substituted alkenyl, halogenated alkenyl, alkynyl, substituted alkynyl, halogenated alkynyl, heterocycloalkyl, substituted heterocycloalkyl, lactam heterocycloalkyl, —CN, —NO₂, —OR₁, —C(O)N(R₁₀)₂,
—NR₁COR₁₆, —N(R₁₀)₂, —SR₁, —S(O)₂R₁, —C(O)R₁₆, —CO₂R₁, aryl, R₇, or R₉;
J, L, M, and Q are N or C(R₆) provided that only one of J, L, M, or Q, is N and the others are C(R₆), further provided that when the core molecule is attached to the pyridinyl moiety at M, Q is C(H), and further provided that there is only one attachment to the core molecule;
G and Y are C(R₆), provided that when the molecule is attached to the phenyl moiety at Y, G is CH;
R₄ is H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, or R₉;
each R₅ is independently H, C₁₋₃ alkyl, or C₂₋₄ alkenyl;
each R₆ is independently H, F, Br, I, Cl, —CN, —CF₃, —OR₅, —SR₅, or —N(R₅)₂, or a bond to the core molecule provided that only one R₆ and no R₃ or R₁₅ is said bond,
V is selected from O, S, or N(R₄);
R₇ is 5-membered heteroaromatic mono-cyclic moieties containing within the ring 1-3 heteroatoms independently selected from the group consisting of —O—, ═N—, —N(R₁₉)—, and —S—, and having 0-1 substituent selected from R₂₀ and further having 0-3 substituents independently selected from F, Cl, Br, or I, or R₇ is a 9-membered fused-ring moiety having a 6-membered ring fused to a 5-membered ring and having the formula 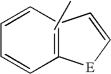
   wherein E is O, S, or NR₁₉, 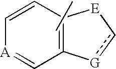
   wherein E and G are independently selected from CR₁₈, O, S, N, or NR₁₉, and A is CR₁₈ or N, or 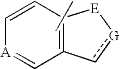
   wherein E and G are independently selected from CR₁₈, O, S, N, or NR₁₉, and A is CR₁₈ or N, each 9-membered fused-ring moiety having 0-1 substituent selected from R₂₀ and further having 0-3 substituent(s) independently selected from F, Cl, Br, or I, and having a bond directly or indirectly attached to the core molecule where valency allows in either the 6-membered or the 5-membered ring of the fused-ring moiety;
each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl, or substituted phenyl;
R₉ is 6-membered heteroaromatic mono-cyclic moieties containing within the ring 1-3 heteroatoms selected from ═N— and having 0-1 substituent selected from R₂₀ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₂₀ and 0-3 substituent(s) independently selected from F, Cl, Br, or I and having a bond directly or indirectly attached to the core molecule where valency allows;
each R¹⁰ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, phenyl, or substituted phenyl;
each R₁₁ is independently H, alkyl, cycloalkyl, heterocyclo-alkyl, halogenated alkyl, halogenated cycloalkyl, or halogenated heterocycloalkyl;
R₁₃ is —OR₁₁, —SR₁₁, —NR₁₁R₁₁, —C(O)R₁₁, —C(O)NR₁₁R₁₁, —CN, —CF₃, —NR₁₁C(O)R₁₁, —S(O)₂NR₁₁R₁₁, —NR₁₁S(O)₂R₁₁, or —NO₂;
each R₁₅ is independently a bond to the core molecule provided that only one R₁₅ and no R₆ or R₃ is also said bond, H, F, Br, Cl, I, alkyl, substituted alkyl, halogenated alkyl, alkenyl, substituted alkenyl, halogenated alkenyl, alkynyl, substituted alkynyl, halogenated alkynyl, heterocycloalkyl, substituted heterocycloalkyl, lactam heterocycloalkyl, —CN, —NO₂, —OR₁, —C(O)N(R₁₀)₂, —NR₁COR₁₆, —N(R₁₀)₂, —SR₁, —CO₂R₁, aryl, R₇, or R₉;
R₁₆ is H, alkyl, substituted alkyl, cycloalkyl, halogenated alkyl, heterocycloalkyl, substituted heterocycloalkyl, substituted phenyl, or substituted naphthyl;
each R₁₈ is independently H, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, —NR₁₁S(O)₂R₁₁, F, Cl, Br, I, or a bond directly or indirectly attached to the core molecule, provided that there is only one said bond to the core molecule within the 9-membered fused-ring moiety, further provided that the fused-ring moiety has 0-1 substituent selected from alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, or —NR₁₁S(O)₂R₁₁, and further provided that the fused-ring moiety has 0-3 substituent(s) selected from F, Cl, Br, or I;
R₁₉ is H, alkyl, halogenated alkyl, substituted alkyl, cycloalkyl, halogenated cycloalkyl, substituted cycloalkyl, phenyl, —SO₂R₈, or phenyl having 1 substituent selected from R₂₀ and further having 0-3 substituents independently selected from F, Cl, Br, or I;
R₂₀ is alkyl, cycloalkyl, heterocycloalkyl, halogenated alkyl, halogenated cycloalkyl, halogenated heterocycloalkyl, —OR₁₁, —SR₁₁, —NR₁₁R₁₁, —C(O)R₁₁, —C(O)NR₁₁R₁₁, —CN, —NR₁₁C(O)R₁₁, —S(O)₂NR₁₁R₁₁, —NR₁₁S(O)₂R₁₁, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
and pharmaceutically acceptable salts thereof. Formula III includes a enantiomers, diastereomers and tautomers.

Examples of compounds of formula III which can be used in the combinations of the present invention are:
N-[(3R)-1-azabicyclo[2.2.2]oct-3-yl]-2,3-dihydrofuro[2,3-c]pyridine-5-carboxamide;
N-[(3R)-1-azabicyclo[2.2.2]oct-3-yl]furo[2,3-c]pyridine-5-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-3-methylfuro[2,3-c]pyridine-5-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
N-[(3R)-1-Azabicyclo[2.2.2]oct-3-yl]furo[3,2-c]pyridine-6-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]thieno[2,3-c]pyridine-5-carboxamide;
N-[(2S,3R)-2-methyl-1-azabicyclo[2.2.2]oct-3-yl]thieno[3,2-c]pyridine-6-carboxamide;
N-[(3S)-1-azabicyclo[2.2.2]oct-3-yl]furo[2,3-c]pyridine-5-carboxamide;
N-[(+/−)1-azabicyclo[2.2.2]oct-3-yl]furo[2,3-c]pyridine-5-carboxamide;
and pharmaceutically acceptable salts thereof.

U.S. Ser. No. 10/262,257, filed Oct. 1, 2002 (U.S. 2003/015359A1, published Aug. 14, 2003), the entire content of which is incorporated herein, describes compounds of the following formula IV and their synthesis which are nicotinic receptor agonists that are useful in the combinations of the subject invention: 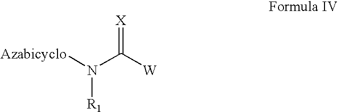
wherein Azabicyclo is

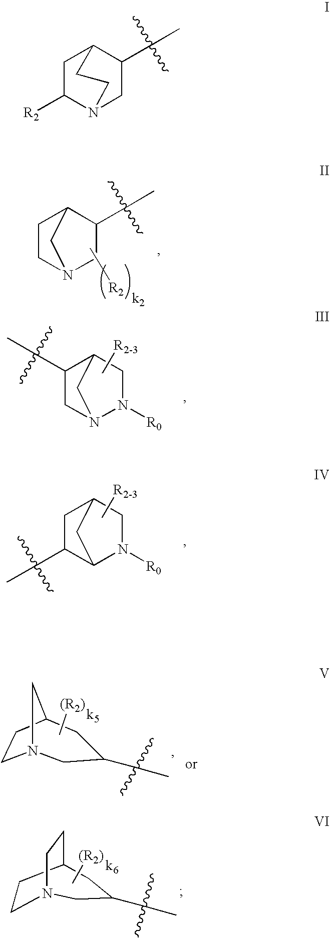

W is 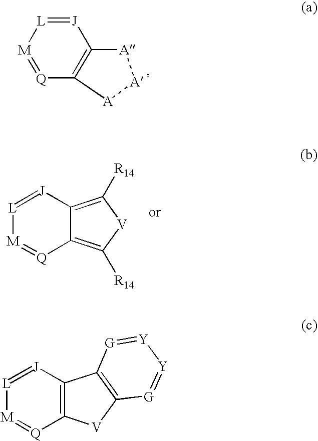
provided that the bond between the —C(═X)— group and the W group may be attached at any available carbon atom within the W group as provided in R₃, R₆, and R₁₅;
X is O, or S;
R₀ is H, lower alkyl, substituted lower alkyl, or halogenated lower alkyl;
each R₁ is H, alkyl, cycloalkyl, halogenated alkyl, substituted phenyl, or substituted naphthyl;
each R₂ is alkyl, halogenated alkyl, substituted alkyl, cycloalkyl, aryl, F, Cl, Br, I, or R₂ is absent provided that k₂, k₅, or k₆ is 0;
R₂₋₃ is H, alkyl, substituted alkyl, halogenated alkyl, F, Cl, Br, or I;
k₂ is 0 or 1;
k₅ and k₆ are independently 0, 1, or 2;
A---A′---A″ is N(R₄)—C(R₃)═C(R₃), N═C(R₃)—C(R₁₅)₂, C(R₃)═C(R₃)—N(R₄), C(R₃)₂—N(R₄)—C(R₃)₂, C(R₁₅)₂—C(R₃)═N, N(R₄)—C(R₃)₂—C(R₃)₂,
C(R₃)₂—C(R₃)₂—N(R₄), O—C(R₃)═C(R₃), O—C(R₃)₂—C(R₃)₂, C(R₃)₂—O—C(R₃)₂, C(R₃)═C(R₃)—O, C(R₃)₂—C(R₃)₂—O, S—C(R₃)═C(R₃), S—C(R₃)₂—C(R₃)₂,
C(R₃)₂—S—C(R₃)₂, C(R₃)═C(R₃)—S, or C(R₃)₂—C(R₃)₂—S;
each R₃ is independently a bond to the core molecule provided that only one R₃ and no R₆ or R₁₅ is also said bond, H, alkyl, substituted alkyl, halogenated alkyl, alkenyl, substituted alkenyl, halogenated alkenyl, alkynyl, substituted alkynyl, halogenated alkynyl, —CN, —NO₂, F, Br, Cl, I, —OR₁₉, —C(O)N(R₁₀)₂, —N(R₁₀)₂, —SR₁₉,
—S(O)₂R₁₉, —C(O)R₁₉, —CO₂R₁₉, aryl, R₇, or R₉;
J, L, M, and Q are N or C(R₆) provided that only one of J, L, M, or Q, is N and the others are C(R₆), further provided that when the core molecule is attached to the pyridinyl moiety at M, Q is C(H), and further provided that there is only one attachment to the core molecule;
G and Y are C(R₆), provided that when the molecule is attached to the phenyl moiety at Y, G is CH;
R₄ is H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, or R₉;
each R₅ is independently H, lower alkyl, or lower alkenyl;
each R₆ is independently H, F, Br, I, Cl, —CN, —CF₃, —OR₅, —SR₅, —N(R₅)₂, or a bond to the core molecule provided that only one R₆ and no R₃ or R₁₅ is said bond;
V is selected from O, S, or N(R₄);
R₇ is 5-membered heteroaromatic mono-cyclic moieties containing within the ring 1-3 heteroatoms independently selected from the group consisting of ═N—, —N(R₁₇)—, —O—, and —S—, and having 0-1 substituent selected from R₁₈ and further having 0-3 substituents independently selected from F, Cl, Br, or I, or R₇ is 9-membered fused-ring moieties having a 6-membered ring fused to a 5-membered ring including the formula 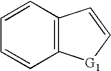
   wherein G₁ is O, S or NR₁₇, 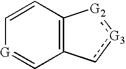
   wherein G is C(R₁₆) or N, and each G₂ and G₃ are independently selected from C(R₁₆)₂, C(R₁₆), O, S, N, and N(R₁₈), provided that both G₂ and G₃ are not simultaneously O, simultaneously S, or simultaneously O and S, or 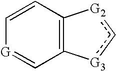
   wherein G is C(R₁₆) or N, and each G₂ and G₃ are independently selected from C(R₁₆)₂, C(R₁₆), O, S, N, and N(R₁₇), each 9-membered fused-ring moiety having 0-1 substituent selected from R₁₈ and further having 0-3 substituent(s) independently selected from F, Cl, Br, or I, wherein the R₇ moiety attaches to other substituents as defined in formula I at any position on either ring as valency allows;
each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl, or substituted phenyl;
R₉ is 6-membered heteroaromatic mono-cyclic moieties containing within the ring 1-3 heteroatoms selected from ═N— and having 0-1 substituent selected from R₁₈ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₁₈ and 0-3 substituent(s) independently selected from F, Cl, Br, or I, and having a bond directly or indirectly attached to the core molecule where valency allows;
each R₁₀ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, phenyl, or substituted phenyl;
each R₁₁ is independently H, alkyl, cycloalkyl, heterocycloalkyl, halogenated alkyl, halogenated cycloalkyl, or halogenated heterocycloalkyl;
R₁₂ is —NO₂, —CN, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁,
—C(O)NR₁₁R₁₁, —NR₁₁C(O)R₁₁, —S(O)₂NR₁₁R₁₁, or —NR₁₁S(O)₂R₁₁;
R₁₃ is —CN, —CF₃, —NO₂, —OR₁₁, —SR₁₁, —NR₁₁R₁₁, —C(O)R₁₁, —C(O)NR₁₁R₁₁,
—NR₁₁C(O)R₁₁, —S(O)₂NR₁₁R₁₁, or —NR₁₁S(O)₂R₁₁;
each R₁₄ is H, alkyl, substituted alkyl, halogenated alkyl, alkenyl, substituted alkenyl, halogenated alkenyl, alkynyl, substituted alkynyl, halogenated alkynyl, F, Br, Cl, I, —CN, —NO₂, —OR₁₉, —C(O)N(R₁₀)₂, —N(R₁₀)₂, —SR₁₉, —S(O)₂R₁₉, —C(O)R₁₉,
—CO₂R₁₉, aryl, R₇ or R₉;
each R₁₅ is independently alkyl, substituted alkyl, halogenated alkyl, alkenyl, substituted alkenyl, halogenated alkenyl, alkynyl, substituted alkynyl, halogenated alkynyl, F, Br, Cl, I, —CN, —NO₂, —OR₁₉, —C(O)N(R₁₀)₂, —N(R₁₀)₂, —SR₁₉, —CO₂R₁₉, aryl, R₇, R₉, or a bond to the core molecule provided that only one R₁₅ and no R₆ or R₃ is said bond;
each R₁₆ is independently H, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, F, Cl, Br, I, —NO₂, —CN, —OR₁₁,
—SR₁₁, —NR₁₁R₁₁, —C(O)R₁₁, —C(O)NR₁₁R₁₁, —NR₁₁C(O)R₁₁, —S(O)₂NR₁₁R₁₁,
—NR₁₁S(O)₂R₁₁, or a bond directly or indirectly attached to the core molecule, provided that there is only one said bond to the core molecule within the 9-membered fused-ring moiety, further provided that the fused-ring moiety has 0-1 substituent selected from alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, or —NR₁₁S(O)₂R₁₁, and further provided that the fused-ring moiety has 0-3 substituent(s) selected from F, Cl, Br, or I;
R₁₇ is H, alkyl, halogenated alkyl, substituted alkyl, cycloalkyl, halogenated cycloalkyl, substituted cycloalkyl, phenyl, —SO₂R₈, or phenyl having 1 substituent selected from R₁₈ and further having 0-3 substituents independently selected from F, Cl, Br, or I;
R₁₈ is alkyl, cycloalkyl, heterocycloalkyl, halogenated alkyl, halogenated cycloalkyl, halogenated heterocycloalkyl, —OR₁₁, —SR₁₁, —NR₁₁R₁₁, —C(O)R₁₁,
—C(O)NR₁₁R₁₁, —CN, —NR₁₁C(O)R₁₁, —S(O)₂NR₁₁R₁₁, —NR₁₁S(O)₂R₁₁, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
R₁₉ is H, alkyl, cycloalkyl, substituted alkyl, halogenated alkyl, substituted phenyl, or substituted naphthyl;
and pharmaceutically acceptable salts thereof. Formula IV encompasses all enantiomers, diastereomers and tautomers.

Examples of compounds of formula IV that can be used in the combinations of the present invention are:
Exo-4(S)-N-(1-azabicyclo[2.2.1]hept-3-yl)furo[2,3-c]pyridine-5-carboxamide;
N-((3R,5R)-1-azabicyclo[3.2.1]oct-3-yl)furo[2,3-c]pyridine-5-carboxamide;
N-[(exo-1-azabicyclo[2.2.1]hept-3-yl]furo[3,2-c]pyridine-6-carboxamide;
N-((3R,5R)-1-azabicyclo[3.2.1]oct-3-yl)furo[3,2-c]pyridine-6-carboxamide;
Exo-4(S)-N-(1-azabicyclo[2.2.1]hept-3-yl)-thieno[2,3-c]pyridine-5-carboxamide;
N-((3R,5R)-1-azabicyclo[3.2.1]oct-3-yl)-thieno[2,3-c]pyridine-5-carboxamide;
Exo-4(S)-N-(1-azabicyclo[2.2.1]hept-3-yl)-thieno[3,2-c]pyridine-6-carboxamide; and
N-((3R,5R)-1-azabicyclo[3.2.1]oct-3-yl)-thieno[3,2-c]pyridine-6-carboxamide;
and pharmaceutically acceptable salts thereof.

For use in medicine, pharmaceutically acceptable salts may be useful in the preparation of the compounds according to the invention. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

Where the nicotinic receptor agonists or antagonists of the invention have at least one asymmetric center, they can accordingly exist as enantiomers. Where the compounds possess two or more asymmetric centers, they can additionally exist as diastereoisomers. It is to be understood that all such isomers and mixtures thereof in any proportion are encompassed within the scope of the present invention.

The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

The pharmaceutically-acceptable cationic salts of nicotinic receptor agonists or antagonists or ziprasidone containing free carboxylic acids can be readily prepared by reacting the free acid form of the nicotinic receptor agonist or antagonist with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate), employing a solvent (e.g., ethyl acetate) from which the desired cationic salt precipitates, or can be otherwise isolated by concentration and/or addition of a non-solvent.

The pharmaceutically acceptable acid addition salts of nicotinic receptor agonists or antagonists or ziprasidone containing free amine groups can be readily prepared by reacting the free base form of the nicotinic receptor agonist or antagonist with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

For the purposes of this specification, Alzheimer's disease is defined in accordance with the NINCDS/ADRDA (National Institute of Neurological and Communicative Disorders and Stroke-Alzheimer's Disease and Related Disorders Association) criteria or DSM-IV criteria.

Although a definitive AD diagnosis requires post-mortem histopathologic confirmation, generally accepted criteria, known as disclosed in the DSM-IV, have been designed for screening, defining and categorizing demented patients. The universally recognized and accepted NINCDS-ADRDA criteria (McKhann et al., 34 Neurology, 939-944; (1984)) can be used in clinical trials to diagnose AD and to evaluate the efficacy of compounds of the present invention. Additionally, the state of the disease before and after treatment can be assessed by various commonly accepted mental-state examinations, including the information-concentration-orientation test (Blessed, 12 Br. J. Psychiatr. Res., 189-198; (1968), the Mini Mental State Examination (MMSE) (Folstein et al., 12 J. Psychiatr. Res., 189-195; (1975)) and the Global Deterioration Scale (Reisberg, 140 Am. J. Psychiatry, 734-739; (1983)).

Psychotic disorders or conditions, such as schizophrenia, schizoaffective disorder, schizophreniform disorder, and schizotypical disorder are conditions in which cognitive enhancement therapy would be beneficial. As provided by the present invention, these psychotic disorders can be treated with a nicotinic receptor agonist or antagonist alone. These agents are especially useful for the apatho-abulic manifestations of schizophrenia and for cognitive impairment of schizophrenia. Psychotic conditions are also treated with atypical antipsychotics. According to the present invention, these conditions can now also be treated with an atypical antipsychotic in combination with, for e.g., varenicline tartrate, a partial nicotine receptor agonist. The atypical antipsychotics can be administered simultaneously with the nicotinic receptor agonists or antagonists, either as separate dosage forms in a kit product, or as one combined dosage form containing both the atypical antipsychotic and the nicotinic receptor agonist or antagonist.

The effects of a pharmaceutical composition comprising an atypical antipsychotic, for example ziprasidone, and a nicotinic receptor agonist or antagonist, of the present invention can be examined by using one or more of the published models of cognition well known in the art.

The pharmaceutical compositions containing an atypical antipsychotic, for example ziprasidone, and a nicotinic receptor agonist or an atypical antipsychotic and a nicotinic receptor antagonist of the present invention are particularly useful for the prevention of, reducing the development of, or reversal of, cognitive impairment disorders and are therefore particularly useful in the treatment of Alzheimer's disease and other dementias. This effect can be demonstrated, for example, by measuring markers such the Reye Auditory Learning Test, Selective Reminding Test, the Weschler Logical Memory Test, and has been shown in clinical studies.

The pharmaceutical compositions containing an atypical antipsychotic, for example ziprasidone, and a nicotinic receptor agonist or antagonist of the present invention are particularly useful for the prevention of, reducing the development of, or reversal of, psychotic disorders, conditions or symptoms and are therefore particularly useful in the treatment of schizophrenia, schizophreniform disorder, schizoaffective disorder or delusional disorder. This can be demonstrated, for example, by measuring markers such Positive or Negative Syndrome Scale (PANSS) and Scales for the Assessment of Negative Symptoms (SANS) or BPRS scores (Kay et al, 13 Schizophrenia Bulletin, 261-276; (1987)), or in various animal models such as PCP or methamphetamine induced locomotor test or the conditioned avoidance response test.

In general, ziprasidone employed in the combinations, pharmaceutical compositions, methods and kits of this invention, will be administered at dosages between about 20 and about 460 mg per day, preferably from about 40 mg to about 200 mg, and most preferably 40 mg to 160 mg together with therapeutically effective amounts of the second therapeutic agent in single or divided doses.

The term “therapeutically effective amount” as used herein refers to a sufficient amount of the compound to treat cognitive impairment disorders and psychotic disorders or conditions at a reasonable risk/benefit ratio applicable to any medical treatment.

The term “treating” as used herein, refers to reversing, alleviating, inhibiting the progress of, or preventing the disorder or condition to which such term applies, or one or more symptoms of such disorder or condition. The term “treatment”, as used herein, refers to the act of “treating” as defined immediately above.

The specific therapeutically effective dose level for any particular patient will depend upon a variety of factors including the disorder being treated and the severity of the disorder; activity of the specific compound employed; the specific composition employed; the age of the patient. However, some variation in dosage may be prescribed depending upon the condition of the subject being treated. The person responsible for administration will, in any event, determine the appropriate dose for the individual subject.

The following dosage amounts and other dosage amounts set forth elsewhere in this description and in the claims are for an average human subject having a weight of about 65 kg to about 70 kg. The skilled practitioner will readily be able to determine the dosage amount required for a subject whose weight falls outside the 65 kg to 70 kg range, based upon the medical history of the subject. All doses set forth herein, and in the claims, are daily doses.

In preferred embodiments, the above nicotinic receptor agonists or antagonists used in the combinations, pharmaceutical compositions, methods and kits of this invention will be administered to treat the conditions described herein in does of about 0.1 milligram to about 1000 milligrams per day. However, some variation in dosage may be prescribed depending upon the condition, age as well as factors, which may alter pharmacokinetics of absorption, distribution, metabolism and excretion in the subject being treated. The person responsible for administration will, in any event, determine the appropriate dose for the individual subject.

One skilled in the art will appreciate that when the nicotine receptor agonist or antagonist is administered to children, the dose may be smaller than the dose that is administered to adults. The exact formulation, route of administration, and dosage can be chosen by the individual physician in view of the patient's condition. Dosage amount and interval can be adjusted individually to provide plasma levels of the active moiety, which are sufficient to maintain therapeutic effects.

It will be recognized by a skilled person that the free base form or other salt forms of the above nicotinic receptor agonists or antagonists can be used in this invention. Calculation of the dosage amount for these other forms of the free base form or other salt forms of a particular nicotinic receptor agonist or antagonist is easily accomplished by performing a simple ratio relative to the molecular weights of the species involved.

The products of the present invention are of use in the treatment and/or prevention of a variety of disorders of the central nervous system. Such disorders include cognitive impairment disorders, such as Alzheimer's disease, age related memory disorder, dementia, including vascular dementia, cognitive impairments caused by traumatic brain injury, dementia due to other general medical conditions (e.g., Human Immunodeficiency Virus disease, head trauma, Parkinson's disease, Huntington's disease), substance-induced persisting dementia (i.e., due to a drug of abuse, a medication, or toxin exposure), dementia due to multiple etiologies, or dementia not otherwise specified, and cognitive disorder not otherwise specified.

The products of the present invention have the advantage that they surprisingly provide greater relief from cognitive impairment and more rapidly than would be expected from administration of either compound alone. The products of the present invention are useful in reducing the complications associated with cognitive impairment disorders.

The meanings attributed to the different types and subtypes of cognitive disorders are as stated in DSM-IV-TR, the contents of which are incorporated by reference herein (“Diagnostic and Statistical Manual of Mental Disorders”, 4^{th} ed, American Psychiatric Assoc., Washington, D.C., 135-181; (2002)).

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; brief psychotic disorder; shared psychotic disorder; psychotic disorder due to a general medical condition; substance-induced psychotic disorder, for example psychosis induced by alcohol, amphetamine, cannabis, cocaine, hallucinogens, inhalants, opioids, or phencyclidine; personality disorder of the paranoid type; personality disorder of the schizoid type; psychotic disorder not otherwise specified.

The meanings attributed to the different types and subtypes of psychotic disorders are as stated in DSM-IV-TR, the contents of which are incorporated by reference herein (“Diagnostic and Statistical Manual of Mental Disorders”, 4^{th} ed, American Psychiatric Assoc., Washington, D.C., 297-343; (2002)).

Schizophrenia as used herein refers to a disorder that lasts for at least 6 months and includes at least one month of active-phase symptoms (i.e., two [or more] of the following: delusions, hallucinations, disorganized speech, grossly disorganized or catatonic behavior, negative symptoms) (“Diagnostic and Statistical Manual of Mental Disorders”, DSM-IV-TR, 4^{th} ed, American Psychiatric Assoc., Washington, D.C., (2002)).

Schizoaffective disorder is defined as a disorder in which a mood episode and the active-phase symptoms of schizophrenia occur together and were preceded or are followed by at least 2 weeks of delusions or hallucinations without prominent mood symptoms (“Diagnostic and Statistical Manual of Mental Disorders”, DSM-IV-TR, 4^{th} ed, American Psychiatric Assoc., Washington, D.C., (2002)).

Schizophreniform disorder is defined as a disorder characterized by a symptomatic presentation that is equivalent to schizophrenia, except for its duration (i.e., the disturbance lasts from 1 to 6 months) and the absence of a requirement that there be a decline in functioning (“Diagnostic and Statistical Manual of Mental Disorders”, DSM-IV-TR, 4^{th} ed, American Psychiatric Assoc., Washington, D.C., (2002)).

Schizotypical disorder is defined as a lifetime pattern of social and interpersonal deficits characterized by an inability to form close interpersonal relationships, eccentric behavior, and mild perceptual distortions.

The combinations of atypical antipsychotics, for example ziprasidone, with nicotine receptor agonists or antagonists in the present invention can be used to treat other psychotic disorders such as delusional disorder; brief psychotic disorder; shared psychotic disorder; substance-induced psychotic disorder, for example psychosis induced by alcohol, amphetamine, cannabis, cocaine, hallucinogens, inhalants, opioids, or phencyclidine; psychotic disorder due to a general medical condition; personality disorder of the paranoid type; personality disorder of the schizoid type; and psychotic disorder not otherwise specified.

For example, “treating schizophrenia, or schizophreniform or schizoaffective disorder” as used herein also encompasses treating one or more symptoms (positive, negative, and other associated features) of said disorders, for example treating, delusions and/or hallucinations associated therewith. Other examples of symptoms of schizophrenia and schizophreniform and schizoaffective disorders include disorganized speech, affective flattening, alogia, anhedonia, inappropriate affect, dysphoric mood (in the form of, for example, depression, anxiety or anger), and some indications of cognitive dysfunction.

Delusional disorder as referred to herein is characterized by at least 1 month of nonbizarre delusions without other active-phase symptoms of schizophrenia. (“Diagnostic and Statistical Manual of Mental Disorders”, DSM-IV-TR, 4^{th} ed., American Psychiatric Assoc., Washington, D.C., (2002)).

Brief psychotic disorder is a disorder that lasts more than 1 day and remits by 1 month. (“Diagnostic and Statistical Manual of Mental Disorders”, DSM-IV-TR, 4^{th} ed., American Psychiatric Assoc., Washington, D.C., (2002)).

Shared psychotic disorder is characterized by the presence of a delusion in an individual who is influenced by someone else who has a longer-standing delusion with similar content. (“Diagnostic and Statistical Manual of Mental Disorders”, DSM-IV-TR, 4^{th} ed., American Psychiatric Assoc., Washington, D.C., (2002)).

Psychotic disorder due to a general medical condition is characterized by psychotic symptoms judged to be a direct physiological consequence of a general medical condition. (“Diagnostic and Statistical Manual of Mental Disorders”, DSM-IV-TR, 4^{th} ed., American Psychiatric Assoc., Washington, D.C., (2002)).

Psychotic disorder not otherwise specified is a psychotic presentation that does not meet the criteria for any of the specific psychotic disorders defined in the DSM-IV-TR (American Psychiatric Assoc., Washington, D.C., (2002)).

In another embodiment, the compounds used in the present invention are useful to treat other disorders that may present with psychotic symptoms as associated features such as dementia of the Alzheimer's type; substance-induced delirium; and major depressive disorder with psychotic features.

In a preferred embodiment, the compounds used in the present invention are useful for treating schizophrenia, a schizoaffective disorder, schizophreniform disorder, or a schizotypical disorder.

The expression “prodrug” refers to compounds that are drug precursors which, following administration, release the drug in vivo via a chemical or physiological process (e.g., a prodrug on being brought to the physiological pH or through enzyme action is converted to the desired drug form).

The present invention includes within its scope the use of prodrugs of ziprasidone, and of nicotinic receptor agonists or antagonists. In general, such prodrugs will be functional derivatives of these compounds which are readily convertible in vivo. Conventional procedures for the selection and preparation of suitable prodrug derivatives are described, for example, in Design of Prodrugs, ed. H. Bundgaard, Elsevier, 1985 and can be achieved using methods well known to those skilled in the art. All such prodrugs are within the scope of the combinations, pharmaceutical compositions, methods and kits of this invention.

The chemist of ordinary skill in the art will also recognize that certain compounds within the scope of this invention can exist in zwitterionic form, i.e., that certain compounds contain an amine portion and a carboxylic acid portion, which, depending upon the pH of the solution, may exist as a free amine and a free carboxylic acid or as a zwitterion in which the amine is protonated to form an ammonium ion and the carboxylic acid is deprotonated to form a carboxylate ion. Use of such zwitterions are included in this invention.

The chemist of ordinary skill in the art will also recognize that some of the compounds of the pharmaceutical combinations contemplated by the present invention can exist in different stereoisomers. Specific stereoisomers may exhibit an ability to treat mental disorders with a more favorable efficacy or safety profile. The present invention includes use of all possible stereoisomers and geometric isomers of the active ingredients of each pharmaceutical combination, and includes not only racemic compounds but also optical isomers as well. In situations where tautomers, i.e. two isomers which are in rapid equilibrium with each other, are possible, the present invention is intended to include use of all tautomeric forms.

The combinations of the present invention can be administered in a standard manner for the treatment of cognitive impairment disorders, psychotic disorders, or mood disorders such as orally, parenterally, transmucosally (e.g., sublingually or via buccal administration), topically, transdermally, rectally, via inhalation (e.g., nasal or deep lung inhalation). Parenteral administration includes, but is not limited to intravenous, intraarterial, intraperitoneal, subcutaneous, intramuscular, intrathecal, and intraarticular, or via a high pressure technique, like Powderject.™

For buccal administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate), or wetting agents (for example, sodium lauryl sulfate). The tablets can be coated according to methods well known in the art.

Such preparations can also be formulated as suppositories for rectal administration, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions of the present invention can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot preparation. Such long acting formulations can be administered by implantation (for example, subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

Solubilized forms of aryl-heterocyclics such as zirpasidone, pharmaceutically acceptable salts there, or prodrugs thereof, or pharmaceutically acceptable salts of prodrugs thereof, associated with (or at levels even greater than) immediate release can be fabricated into depot formulations. For example, a pharmaceutical kit comprising ziprasidone, ziprasidone salts or prodrugs thereof, or pharmaceutically acceptable salts of ziprasidone prodrugs, which can be solubilized or unsolubilized; and a constituting liquid vehicle comprised of a viscosity agent with the proviso that when the ziprasidone compound is unsolubilized, the aqueous liquid further comprises a solubilizer.

Ziprasidone depot formulation in the form of a suspension are described in U.S. Patent Application Ser. No. 60/421,295, filed Oct. 25, 2002, which is incorporated herein by reference in its entirety. Novel injectable depot formulations of ziprasidone are described in U.S. Patent Application Ser. No. 60/421,473, filed Oct. 25, 2002, which are incorporated herein by reference in its entirety.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols.

Alternatively, the compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The combinations of this invention can also be administered in a controlled release formulation such as a slow release or a fast release formulation. Such controlled release formulations of the combinations of this invention may be prepared using methods well known to those skilled in the art. The method of administration will be determined, by the attendant physician or other person skilled in the art after an evaluation of the patient's condition and requirements.

By the term “controlled release” is meant release of the active substance from the dosage form is modified to occur at a slower rate than that from an immediate release product, such as a conventional swallow tablet or capsule.

By the term “immediate release” is meant a pharmaceutical composition in which one of more active ingredients therein demonstrates at least about 80-100% (w/v) dissolution, preferably between from about 90% (w/v) to about 95% (w/v) within about 15 to 20 minutes as determined by a standard dissolution test. Suitable standard dissolution tests are known in the field.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain an atypical antipsychotic in sustained or controlled release form and, a second therapeutic agent in an immediate release form. Alternatively, the atypical antipsychotic can be in immediate release form and the second therapeutic agent can be in sustained or controlled release form.

The combinations of this invention can also be administered in parenteral form. For parenteral administration, solutions in sesame or peanut oil or in aqueous propylene glycol can be employed, as well as sterile aqueous solutions of the corresponding water-soluble salts. Such aqueous solutions can be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art. For examples, methods of preparing pellets are described in Remington: The Science and Practice of Pharmacy, Mack Publishing Company, Easton, Pa., 19th Edition (1995). Prolonged release pellets are prepared by either coating immediate release pellets or via matrix systems. Coating may be carried out, for example, in coating pans or in fluid bed coater-driers. Extrusion and subsequent spheronization is a long-known method for the preparation of pharmaceutical pellets (J. W. Conine et al., Drug & Cosmetic Ind. 106: 38-41; (1970)). However, other methods such as pelletization may be utilized. Particles may be agglomerated to form spherical granules or pellets, in a high speed mixer granulator, or rotary fluid bed agglomerator. These methods are described by K. W. Olson and A. M. Mehta, Int. J. Pharm. Tech & Prod. Mfr., 6: 18-24; (1985). Pellets may be also prepared by extrusion of wet masses or melts followed by spheronisation, for example as described in C. Vervaet, L. Baert & J. P. Remon, Int. J. Pharm. 116: 131-146; (1995). Excipients used are typically those with plastic qualities such as microcrystalline cellulose, but also mannitol. Small quantities of a polymeric binder are generally added. Surfactants such as sodium dodecyl sulphate may also be incorporated to give easier extrusion.

Pharmaceutical compositions according to the invention can contain 0.1%-95% of the therapeutic agents of this invention, preferably 1%-70%. In any event, the composition or formulation to be administered will contain a quantity of therapeutic agent(s) according to the invention in an amount effective to treat the condition or disease of the subject being treated.

The two different active ingredients of the compositions of this invention can be co-administered simultaneously or sequentially in any order, or as a single pharmaceutical composition comprising, for example, ziprasidone and a nicotinic receptor agonist or antagonist as described above.

Since the present invention has an aspect that relates to the treatment of the disease/conditions described herein with a combination of active ingredients, which can be administered separately, the invention also relates to combining separate pharmaceutical compositions in kit form. The kit comprises two separate pharmaceutical compositions: ziprasidone and a nicotinic receptor agonist or antagonist, a prodrug thereof or a pharmaceutically acceptable salt of said nicotinic receptor agonist or antagonist or prodrug. The kit includes a container for containing the separate compositions such as a divided bottle or a divided foil packet. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . . Second Week, Monday, Tuesday, . . . ” etc. Other variations of memory aids will be readily apparent to the skilled practitioner. A “daily dose” can be a single tablet or capsule or several pills or capsules to be taken on a given day. Also, a daily dose of the ziprasidone can consist of one tablet or capsule while a daily dose of the nicotinic receptor agonist or antagonist can consist of several tablets or capsules or vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter, which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

It will be understood that while the use of a single atypical antipsychotic as a first component compound is preferred, combinations of two or more atypical antipsychotics can be used as a first component if necessary or desired. Similarly, while the use of a single nicotinic receptor agonist or antagonist as a second component compound is preferred, combinations of two or more of these agents can be used as a second component if necessary or desired.

The atypical antipsychotic of the present invention is useful alone or in combination with a second antipsychotic agent, for example, an atypical antipsychotic such as ziprasidone mesylate, a typical antipsychotic such as haloperidol, or a dopamine system stabilizer antipsychotic such as aripiprazole. It is preferred that if a second antipsychotic agent is used that they both administered to the patient in synergistic effective amounts. It is preferred that the total amount ranges from about 0.0001 to about 1000 mg/kg per day, more preferably from about 0.01 to about 100 mg/kg per day and most preferably from about 0.1 to about 60 mg/kg per day.

Pharmaceutical compositions of use in the present invention will contain one or both active compound(s) in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampules, auto-injector devices or suppositories; for oral, parenteral, intranasal, sublingual or rectal administration, or for administration by inhalation or insufflation. For preparing solid compositions such as tablets, the principal active ingredients are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof.

When referring to these preformulation compositions as “homogeneous”, it is meant that the active ingredients are dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 2000 mg of each of the active ingredients of the present invention. Typical unit dosage forms contain from 1 to 300 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

When administered in combination, either as a single or as separate pharmaceutical composition(s), the ziprasidone and the nicotinic receptor agonist or antagonist are presented in a ratio which is consistent with the manifestation of the desired effect. In particular, the ratio by weight of ziprasidone to the nicotinic receptor agonist or antagonist will suitably be between 0.001 to 1 and 1000 to 1, and especially between 0.01 to 1 and 100 to 1.

The pharmaceutical combinations can be administered on a regimen of up to 6 times per day, preferably 1 to 4 times per day, especially 2 times per day, and most especially once daily.

As used herein the term “mammal” includes animals of economic importance such as bovine, and porcine animals, especially those that produce meat, as well as domestic animals (e.g. cats and dogs), sports animals (e.g. horses), zoo animals, and humans, the latter being most preferred.

### EXAMPLE 1

A pharmaceutical composition is prepared by combining ziprasidone with a nicotinic receptor agonist, which is varenicline tartrate, in a pharmaceutically acceptable carrier. The composition contains respective amounts of ziprasidone and varenicline tartrate to deliver on a daily basis between about 20 mg to about 160 mg ziprasidone and a therapeutically effective amount of the nicotinic receptor agonist or antagonist. The composition is administered to a patient for the treatment of schizophrenia and/or cognitive impairment on a daily, twice daily, three times daily, or four times daily basis.

It should be understood that the invention is not limited to the particular embodiments and examples described herein, but that various changes and modifications may be made without departing from the spirit and scope of this novel concept as defined by the following claims.

1. A pharmaceutical composition for treating cognitive impairment or a psychotic disorder in a mammal comprising (a) an amount of a first therapeutic agent which is an atypical antipsychotic and (b) an amount of a second therapeutic agent which is a nicotinic receptor agonist or antagonist, wherein the amounts of (a) and (b) are together effective in treating the cognitive impairment or psychotic disorder.

2. The pharmaceutical composition of claim 1 where the first therapeutic agent is selected from the group consisting of olanzapine, aripiprazole, clozapine, risperidone, sertindole, quetiapine, amisulpride, ziprasidone, asenapine, paliperidone, bifeprunox, and pharmaceutically acceptable salts of any of the foregoing.

3. The pharmaceutical composition of claim 1, wherein the first therapeutic agent is ziprasidone or a pharmaceutically acceptable salt thereof.

4. The pharmaceutical composition of claim 1, wherein the first therapeutic agent is ziprasidone or a pharmaceutically acceptable salt thereof, and the second agent is varenicline or a pharmaceutically acceptable salt thereof.

5. The pharmaceutical composition of claim 1, wherein the first therapeutic agent is ziprasidone or a pharmaceutically acceptable salt thereof, and the second agent is a compound of formula I 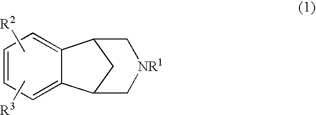
wherein R¹ is hydrogen, (C₁-C₆)alkyl, unconjugated (C₃-C₆)alkenyl, benzyl, XC(═O)R¹³ or —CH₂CH₂—O—(C₁-C₄)alkyl;
R² and R³ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁₋C₆)alkylamino-, [(C₁-C₆)alkyl]₂amino-, —CO₂R⁴, —CONR⁵R⁶, —SO₂NR⁷R⁸, —C(═O)R¹³, —XC(═O)R¹³, aryl-(C₀-C₃)alkyl- or aryl-(C₀-C₃)alkyl-O—, wherein said aryl is selected from phenyl and naphthyl, heteroaryl-(C₀-C₃)alkyl- or heteroaryl-(C₀-C₃)alkyl-O—, wherein said heteroaryl is selected from five to seven membered aromatic rings containing from one to four heteroatoms selected from oxygen, nitrogen and sulfur; X²(C₀-C₆)alkyl- and X²(C₁-C₆)alkoxy-(C₀-C₆)alkyl-, wherein X² is absent or X² is (C₁-C₆)alkylamino- or [(C₁-C₆)alkyl]₂amino-, and wherein the (C₀-C₆)alkyl- or (C₁-C₆)alkoxy-(C₀-C₆)alkyl- moieties of said X²(C₀-C₆)alkyl- or X²(C₁-C₆)alkoxy-(C₀-C₆)alkyl- contains at least one carbon atom, and wherein from one to three of the carbon atoms of said (C₀-C₆)alky- or (C₁-C₆)alkoxy-(C₀-C₆)alkyl- moieties may optionally be replaced by an oxygen, nitrogen or sulfur atom, with the proviso that any two such heteroatoms must be separated by at least two carbon atoms, and wherein any of the alkyl moieties of said (C₀-C₆)alkyl- or (C₁₋C₆)alkoxy-(C₀-C₆)alkyl- groups may be optionally substituted with from two to seven fluorine atoms, and wherein one of the carbon atoms of each of the alkyl moieties of said aryl-(C₀-C₃)alkyl- and said heteroaryl-(C₀-C₃)alkyl- may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluorine atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)alkylamino-, [(C₁-C₆)alkyl]₂amino-, —CO₂R⁴, —CONR⁵R⁶, —SO₂NR⁷R⁸, —C(═O)R¹³ and —XC(═O)R¹³;
or R² and R³, together with the carbons to which they are attached, form a four to seven membered monocyclic, or a ten to fourteen membered bicyclic, carbocyclic ring that can be saturated or unsaturated, wherein from one to three of the non-fused carbon atoms of said monocyclic rings, and from one to five of the carbon atoms of said bicyclic rings that are not part of the benzo ring shown in formula I, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkyl- or (C₁-C₆)alkoxy-(C₀-C₆)alkyl-, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino-, [(C₁-C₆)alkyl]₂amino-, —CO₂R⁴, —CONR⁵R⁶, —SO₂NR⁷R⁸, —C(═O)R¹³, and —XC(═O)R¹³;
each R⁴, R⁵, R⁶, R⁷, R⁸ and R¹³ is selected, independently, from hydrogen and (C₁-C₆) alkyl, or R⁵ and R⁶, or R⁷ and R⁸ together with the nitrogen to which they are attached, form a pyrrolidine, piperidine, morpholine, azetidine, piperazine, —N-(C₁-C₆)alkylpiperazine or thiomorpholine ring, or a thiomorpholine ring wherein the ring sulfur is replaced with a sulfoxide or sulfone; and
each X is, independently, (C₁-C₆)alkylene;
with the proviso that: (a) at least one of R¹, R² and R³ must be the other than hydrogen, and (b) when R² and R³ are hydrogen, R¹ cannot be hydrogen, (C₁-C₆)alkyl, or unconjugated (C₃-C₆)alkenyl;
or a pharmaceutically acceptable salt thereof.

6. The pharmaceutical composition of claim 5, wherein the compound of formula I is selected from:
5,13-diazatetracyclo[9.3.1.0^{2,10}.0^{4,8}]pentadeca-2,4(8),9-trien-6-one;
6-oxo-5-oxa-7,13-diazatetracyclo[9.3.1.0^{2,10}.0^{4,8}]pentadeca-2(10),3,6,8-tetraene;
2-fluoro-N-(4-hydroxy-10-aza-tricyclo[6.3.1.0^{2,7}]dodeca-2(7),3,5-trien-5-yl)benzamide;
6-methyl-5-thia-7,13-diazatetracyclo[9.3.1.0^{2,10}.0^{4,8}]pentadeca-2(10),3,6,8-tetraene;
6-methyl-7-propyl-5,7,13-triazatetracyclo[9.3.1.0^{2,10}.0^{4,8}]pentadeca-2(10),3,5,8-tetraene;
5,7,13-triazatetracyclo[9.3.1.0^{2,10}.0^{4,8}]pentadeca-2(10),3,5,8-tetraene;
7-methyl-5,7,13-triazatetracyclo[9.3.1.0^{2,10}.0^{4,8}]pentadeca-2(10),3,5,8-tetraene;
6-methyl-5,7,13-triazatetracyclo[9.3.1.0^{2,10}.0^{4,8}]pentadeca-2(10),3,5,8-tetraene;
6,7-dimethyl-5,7,13-triazatetracyclo[9.3.1.0^{2,10}.0^{4,8}]pentadeca-2(10),3,5,8-tetraene;
7-propyl-5,7,13-triazatetracyclo[9.3.1.0^{2,10}.0^{4,8}]pentadeca-2(10),3,5,8-tetraene;
7-butyl-5,7,13-triazatetracyclo[9.3.1.0^{2,10}.0^{4,8}]pentadeca-2(10),3,5,8-tetraene;
6-methyl-7-isobutyl-5,7,13-triazatetracyclo[9.3.1.0^{2,10}.0^{4,8}]pentadeca-2(10),3,5,8-tetraene;
7-phenyl-5,7,13-triazatetracyclo[9.3.1.0^{2,10}.0^{4.8}]pentadeca-2(10),3,5,8-tetraene;
6-methyl-7-phenyl-5,7,13-triazatetracyclo[9.3.1.0^{2,10}.0^{4,8}]pentadeca-2(10),3,5,8-tetraene;
7-neopentyl-5,7,13-triazatetracyclo[9.3.1.0^{2,10}.0^{4,8}]pentadeca-2(10),3,5,8-tetraene;
6-methyl-7-neopentyl-5,7,13-triazatetracyclo[9.3.1.0^{2,10}.04^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
14-methyl-5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
7-methyl-5-oxa-6,13-diazatetracyclo[9.3.1.0^{2,10}.0^{4,8}]pentadeca-2,4(8),6,9-tetraene;
4-methyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
4-amino-10-azatricyclo[6.3.1.0^{2,7}]dodeca-2(7),3,5-triene;
N′-[10-azatricyclo[6.3.1.0^{2,7}]dodeca-2(7),3,5-trien-4-yl]acetamide;
4,5-dinitro-10-aza-tricyclo[6.3.1.0^{2,7}]dodeca-2(7),3,5-triene;
4,5-difluoro-10-aza-tricyclo[6.3.1.0^{2,7}]dodeca-2(7),3,5-triene;
4-chloro-10-azatricyclo[6.3.1.0^{2,7}]dodeca-2(7),3,5-triene;
3-(10-azatricyclo[6.3.1.0^{2,7}]dodeca-2(7),3,5-trien-4-yl)-5-methyl-1,2,4-oxadiazole;
10-azatricyclo[6.3.1.0^{2,7}]dodeca-2(7),3,5-trien-4-ol;
4,5-dichloro-10-azatricyclo[6.3.1.0^{2,7}]dodeca-2(7),3,5-triene;
N⁴,N⁴-dimethyl-10-azatricyclo[6.3.1.0^{2,7}]dodeca-2(7),3,5-triene-4-sulfonamide;
4-(1-pyrrolidinylsulfonyl)-10-azatricyclo[6.3.1.0^{2,7}]dodeca-2(7),3,5-triene;
1-(10-azatricyclo[6.3.1.0^{2,7}]dodeca-2(7),3,5-trien-4-yl)-1-ethanone;
3-trifluoromethyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
3-fluoro-10-aza-tricyclo[6.3.1.0^{2,7}]dodeca-2(7),3,5-triene;
10-azatricyclo[6.3.1.0^{2,7}]dodeca-2(7),3,5-trien-4-yl cyanide;
4-fluoro-10-aza-tricyclo[6.3.1.0^{2,7}]dodeca-2(7),3,5-triene;
5,14-diazatetracyclo[10.3.1.0^{2,11}.0^{4,9}]hexadeca-2(11),3,5,7,9-pentaene;
6-methyl-5,14-diazatetracyclo[10.3.1.0^{2,11}.0^{4,9}]hexadeca-2 (11),3,5,7,9-pentaene;
7-methyl-5,14-diazatetracyclo[10.3.1.0^{2,11}.0^{4,9}]hexadeca-2(11),3,5,7,9-pentaene;
7-ethyl-5,14-diazatetracyclo[10.3.1.0^{2,11}.0^{4,9}]hexadeca-2(11),3,5,7,9-pentaene;
8-methyl-5,14-diazatetracyclo[10.3.1.0^{2,11}.0^{4,9}]hexadeca-2(11),3,5,7,9-pentaene;
5,14-diazatetracyclo[10.3.1.0^{2,11}.0^{4,9}]hexadeca-2(11),3,7,9-tetraen-6-one;
6-chloro-5,14-diazatetracyclo[10.3.1.0^{2,11}.0^{4,9}]hexadeca-2(11),3,5,7,9-pentaene;
6-methoxy-5,14-diazatetracyclo[10.3.1.0^{2,11}.0^{4,9}]hexadeca-2(11),3,5,7,9-pentaene;
6-chloro-10-fluoro-5,14-diazatetracyclo[10.3.1.0^{2,11}.0^{4,9}-]hexadeca-2(11),3,5,7,9-pentaene;
5,8,14-triazatetracyclo[10.3.1.0^{2,11}.0^{4,9}]hexadeca-2(11),3,7,9-tetraen-6-one;
6-chloro-3-fluoro-5,14-diazatetracyclo[10.3.1.0^{2,11}.0^{4,9}]hexadeca-2(11),3,5,7,9-pentaene;
6-methyl-5,7-dioxo-6,13-diazatetracyclo[9.3.1.0^{2,10}.0^{4,8}]pentadeca-2(10),3,8-triene;
6-methyl-5-oxo-6,13-diazatetracyclo[9.3.1.0^{2,10}.0^{4,8}]pentadeca-2(10),3,8-triene;
5,7-dimethyl-6-oxo-5,7,13-triazatetracyclo[9.3.1.0^{2,10}.0^{4,8}]pentadeca-2(10),3,8-triene;
5,7-dioxo-6,13-diazatetracyclo[9.3.1.0^{2,10}.0^{4,8}]pentadeca-2 (10),3,8-triene;
5-oxo-6,13-diazatetracyclo[9.3.1.0^{2,10}.0^{4,8}]pentadeca-2(10),3,8-triene;
6-oxo-5,7,13-triazatetracyclo[9.3.1.0^{2,10}.0^{4,8}]pentadeca-2(10),3,8-triene;
6-methyl-5-thia-5-dioxo-6,13-diazatetracyclo[9.3.1.0^{2,10}.0^{4,8}]pentadeca-2(10),3,6,8-tetraene;
7-dimethylamino-5-thia-5-dioxo-6,13-diazatetracyclo[9.3.1.0^{2,10}.0^{4,8}]pentadeca-2(10),3,6,8-tetraene;
6,7-dioxo-5,8,14-triazatetracyclo[10.3.1.0^{2,11}.0^{4,9}]hexadeca-2(11),3,9-triene;
5,8-dimethyl-6,7-dioxo-5,8,14-triazatetracyclo[10.3.1.0^{2,11}.0^{4,9}]hexadeca-2(11),3,9-triene;
5-oxa-7-methyl-6-oxo-7,13-diazatetracyclo[9.3.1.0^{2,10}.0^{4,8}]pentadeca-2(10),3,8-triene;
5-fluoro-10-aza-tricyclo[6.3.1.0^{2,7}]dodeca-2(7),3,5-triene-4-carbonitrile;
4-ethynyl-5-fluoro-10-aza-tricyclo[6.3.1.0^{2,7}]dodeca-2(7),3,5-triene;
5-ethynyl-10-aza-tricyclo[6.3.1.0^{2,7}]dodeca-2(7),3,5-triene-4-carbonitrile;
5-chloro-10-aza-tricyclo[6.3.1.0^{2,7}]dodeca-2(7),3,5-triene-4-carbon itrile;
4-ethynyl-5-chloro-10-aza-tricyclo[6.3.1.0^{2,7}]dodeca-2(7),3,5-triene;
4-fluoro-5-trifluoromethyl-10-aza-tricyclo[6.3.1.0^{2,7}]dodeca-2(7),3,5-triene;
4-chloro-5-trifluoromethyl-10-aza-tricyclo[6.3.1.0^{2,7}]dodeca-2(7),3,5-triene;
5-trifluoromethyl-10-aza-tricyclo[6.3.1.0^{2,7}]dodeca-2(7),3,5-triene-4-carbonitrile;
4-ethynyl-5-trifluoromethyl-10-aza-tricyclo[6.3.1.0^{2,7}]dodeca-2(7),3,5-triene; and
4,5-bistrifluoromethyl-10-aza-tricyclo[6.3.1.0^{2,7}]dodeca-2(7),3,5-triene;
and pharmaceutically acceptable salts thereof.

7. The pharmaceutical composition of claim 1, wherein the first therapeutic agent is ziprasidone or a pharmaceutically acceptable salt thereof and the second therapeutic agent is an alpha 7 subtype selective nicotinic receptor agonist.

8. The pharmaceutical composition of claim 1, wherein the first therapeutic agent is ziprasidone or a pharmaceutically acceptable salt thereof and the second therapeutic agent is a compound of formula II 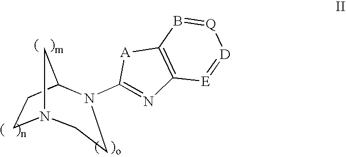
wherein
n=1-2;
m=1-2;
o 1-2;
A O, S or NR¹;
B N or CR²;
Q=N or CR³;
D=N or CR⁴;
E=N or CR⁵;
R¹ is H, a straight chain or branched (C₁-C₈)alkyl, C(═O)OR⁶, CH₂R⁶, C(═O)NR⁶R⁷, C(═O)R⁶, or SO₂R⁶;
each R², R³, R⁴ and R⁵ is independently selected from F, Cl, Br, I, nitro, cyano, CF₃, —NR⁶R⁷, —NR⁶C(═O)R⁷, —NR⁶C(═O)NR⁷R⁸, —NR⁶C(═O)OR⁷, —NR⁶S(═O)₂R⁷—, NR⁶S(═O)₂NR⁷R⁸, —OR⁶, —OC(═O)R⁶, —OC(═O)OR⁶, —OC(═O)NR⁶R⁷, —OC(═O)SR⁶, —C(═O)OR⁶, —C(═O)R⁶, —C(═O)NR⁶R⁷, —SR⁶, —S(═O)R⁶, —S(═O)₂R⁶, —S(═O)₂NR⁶R⁷, and R⁶;
each R⁶, R⁷, and R⁸ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, 5-11 membered heterobicycloalkenyl, (C₆-C₁₁)aryl, and 5-12 membered heteroaryl; wherein each R⁶, R⁷, and R⁸ is optionally substituted with from one to six substituents, independently selected from F, Cl, Br, I, nitro, cyano, CF₃, —NR⁹R¹⁰, —NR⁹C(═O)R¹⁰, —NR⁹C(═O)NR¹⁰R¹¹, —NR⁹C(═O)OR¹⁰, —NR⁹S(═O)₂R₁₀, —NR⁹S(═O)₂NR¹⁰R¹¹, —OR⁹, —OC(═O)R⁹, —OC(═O)OR⁹, —OC(═O)NR⁹R¹⁰, —OC(═O)SR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹;
each R⁹, R¹⁰ and R¹¹ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, (5-11 membered) heterobicycloalkenyl, (C₆-C₁₁)aryl or 5-12 membered heteroaryl; wherein each R⁹, R¹⁰ and R¹¹ is optionally substituted with from one to six substituents independently selected from F, Cl, Br, I, nitro, cyano, CF₃, —NR¹²R¹³, —NR¹²C(═O)R¹³, —NR¹²C(═O)NR¹³R¹⁴, —NR¹²C(═O)OR¹³, —NR¹²S(═O)₂R¹³, —NR¹²S(═O)₂NR¹³R¹⁴, —OR¹², —OC(═O)R¹², —OC(═O)OR¹²—OC(═O)NR¹²R¹³, —OC(═O)SR¹², —C(═O)OR¹², —C(═O)R¹², —C(═O)NR¹²R¹³, —SR¹², —S(═O)R¹², —S(═O)₂R¹², —S(═O)₂NR¹²R¹³ and R¹²;
each R¹², R¹³, and R¹⁴ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, 5-11 membered heterobicycloalkenyl, (C₆-C₁₁)aryl and (5-12 membered) heteroaryl;
or R² and R³, or R³ and R⁴, or R⁵ and R⁵, may form another 6-membered aromatic or heteroaromatic ring sharing B and Q, or Q and D, or D and E, respectively, and may be optionally substituted with from one to four substitutuents independently selected from the group of radicals set forth in the definition of R⁶, R⁷ and R⁸ above;
or a pharmaceutically acceptable salts thereof.

9. The pharmaceutical composition of claim 8, wherein the compound of formula II is selected from:
4-oxazolo[5,4-b]pyridin-2-yl-1,4-diazabicyclo[3.2.2]nonane;
4-oxazolo[5,4-c]pyridin-2-yl-1,4-diazabicyclo[3.2.2]nonane;
4-oxazolo[4,5-c]pyridin-2-yl-1,4-diazabicyclo[3.2.2]nonane;
4-oxazolo[4,5-b]pyridin-2-yl-1,4-diazabicyclo[3.2.2]nonane;
4-(5-methyl-oxazolo[4,5-b]pyridin-2-yl)-1,4-diazabicyclo[3.2.2]nonane;
4-(6-phenyl-oxazolo[5,4-b]pyridin-2-yl)-1,4-diazabicyclo[3.2.2]nonane;
4-(6-bromo-oxazolo[4,5-b]pyridin-2-yl)-1,4-diazabicyclo[3.2.2]nonane; and
4-(6-phenyl-oxazolo[4,5-b]pyridin-2-yl)-1,4-diazabicyclo[3.2.2]nonane;
and pharmaceutically acceptable salts thereof.

10. The pharmaceutical composition of claim 1, wherein the first therapeutic agent is ziprasidone or a pharmaceutically acceptable salt thereof and the second therapeutic agent is a compound of formula III 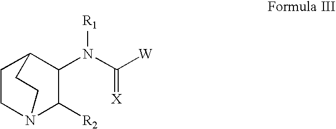
wherein W is 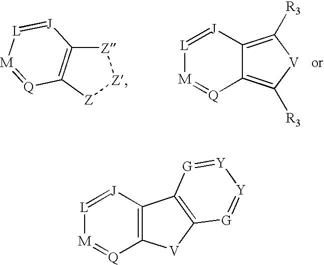
provided that the bond between the —C(═X)— group and the W group may be attached at any available carbon atom within the W group as provided in R₃, R₆, and R₁₅;
X is O, or S;
each R₁ is H, alkyl, cycloalkyl, halogenated alkyl, substituted phenyl, or substituted naphthyl;
R₂ is H, alkyl, halogenated alkyl, substituted alkyl, cycloalkyl, or aryl;
Z---Z′---Z″ is selected from N(R₄)—C(R₃)═C(R₃), N═C(R₃)—C(R₁₅)₂, C(R₃)═C(R₃)—N(R₄), C(R₃)₂—N(R₄)—C(R₃)₂, C(R₁₅)₂—C(R₃)═N, N(R₄)—C(R₃)₂—C(R₃)₂, C(R₃)₂—C(R₃)₂—N(R₄), O—C(R₃)═C(R₃), O—C(R₃)₂—C(R₃)₂, C(R₃)₂—O—C(R₃)₂, C(R₃)═C(R₃)—O, C(R₃)₂—C(R₃)₂—O, S—C(R₃)═C(R₃), S—C(R₃)₂—C(R₃)₂, C(R₃)₂—S—C(R₃)₂, C(R₃)═C(R₃)—S, or C(R₃)₂—C(R₃)₂—S;
each R₃ is independently a bond to the core molecule provided that only one R₃ and no Rr, or R₁₅ is also said bond, H, F, Br, Cl, I, alkyl, substituted alkyl, halogenated alkyl, alkenyl, substituted alkenyl, halogenated alkenyl, alkynyl, substituted alkynyl, halogenated alkynyl, heterocycloalkyl, substituted heterocycloalkyl, lactam heterocycloalkyl, —CN, —NO₂, —OR₁, —C(O)N(R₁₀)₂,
—NR₁COR₁₆, —N(R₁₀)₂, —SR₁, —S(O)₂R₁, —C(O)R₁₆, —CO₂R₁, aryl, R₇, or R₉;
J, L, M, and Q are N or C(R₆) provided that only one of J, L, M, or Q, is N and the others are C(R₆), further provided that when the core molecule is attached to the pyridinyl moiety at M, Q is C(H), and further provided that there is only one attachment to the core molecule;
G and Y are C(Rr), provided that when the molecule is attached to the phenyl moiety at Y, G is CH;
R₄ is H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, or R₉;
each R₅ is independently H, C₁₋₃ alkyl, or C₂₋₄ alkenyl;
each R₆ is independently H, F, Br, I, Cl, —CN, —CF₃, —OR₅, —SR₅, or —N(R₅)₂, or a bond to the core molecule provided that only one R₆ and no R₃ or R₁₅ is said bond, V is selected from O, S, or N(R₄);
R₇ is 5-membered heteroaromatic mono-cyclic moieties containing within the ring 1-3 heteroatoms independently selected from the group consisting of —O—, ═N—, —N(R₁₉)—, and —S—, and having 0-1 substituent selected from R₂₀ and further having 0-3 substituents independently selected from F, Cl, Br, or I, or R₇ is a 9-membered fused-ring moiety having a 6-membered ring fused to a 5-membered ring and having the formula 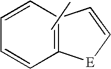
wherein E is O, S, or NR₁₉, 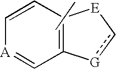
wherein E and G are independently selected from CR₁₈, O, S, N, or NR₁₉, and A is CR₁₈ or N, or 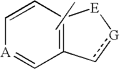
wherein E and G are independently selected from CR₁₈, O, S, N, or NR₁₉, and A is CR₁₈ or N, each 9-membered fused-ring moiety having 0-1 substituent selected from R₂₀ and further having 0-3 substituent(s) independently selected from F, Cl, Br, or I, and having a bond directly or indirectly attached to the core molecule where valency allows in either the 6-membered or the 5-membered ring of the fused-ring moiety;
each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl, or substituted phenyl;
R₉ is 6-membered heteroaromatic mono-cyclic moieties containing within the ring 1-3 heteroatoms selected from ═N— and having 0-1 substituent selected from R₂₀ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₂₀ and 0-3 substituent(s) independently selected from F, Cl, Br, or I and having a bond directly or indirectly attached to the core molecule where valency allows;
each R₁₀ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, phenyl, or substituted phenyl;
each R₁₁ is independently H, alkyl, cycloalkyl, heterocyclo-alkyl, halogenated alkyl, halogenated cycloalkyl, or halogenated heterocycloalkyl;
R₁₃ is —OR₁₁, —SR₁₁, —NR₁₁R₁₁, —C(O)R₁₁, —C(O)NR₁₁R₁₁, —CN, —CF₃, —NR₁₁C(O)R₁₁, —S(O)₂NR₁₁R₁₁, —NR₁₁S(O)₂R₁₁, or —NO₂;
each R₁₅ is independently a bond to the core molecule provided that only one R₁₅ and no R⁶ or R₃ is also said bond, H, F, Br, Cl, I, alkyl, substituted alkyl, halogenated alkyl, alkenyl, substituted alkenyl, halogenated alkenyl, alkynyl, substituted alkynyl, halogenated alkynyl, heterocycloalkyl, substituted heterocycloalkyl, lactam heterocycloalkyl, —CN, —NO₂, —OR₁, —C(O)N(R₁₀)₂, —NR₁COR_{,16}, —N(R₁₀)₂, —SR₁, —CO₂R₁, aryl, R₇, or R₉;
R₁₆ is H, alkyl, substituted alkyl, cycloalkyl, halogenated alkyl, heterocycloalkyl, substituted heterocycloalkyl, substituted phenyl, or substituted naphthyl;
each R₁₈ is independently H, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, —NR₁₁S(O)₂R₁₁, F, Cl, Br, I, or a bond directly or indirectly attached to the core molecule, provided that there is only one said bond to the core molecule within the 9-membered fused-ring moiety, further provided that the fused-ring moiety has 0-1 substituent selected from alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, or —NR₁₁S(O)₂R₁₁, and further provided that the fused-ring moiety has 0-3 substituent(s) selected from F, Cl, Br, or I;
R₁₉ is H, alkyl, halogenated alkyl, substituted alkyl, cycloalkyl, halogenated cycloalkyl, substituted cycloalkyl, phenyl, —SO₂R₈, or phenyl having 1 substituent selected from R₂₀ and further having 0-3 substituents independently selected from F, Cl, Br, or I;
R₂₀ is alkyl, cycloalkyl, heterocycloalkyl, halogenated alkyl, halogenated cycloalkyl, halogenated heterocycloalkyl, —OR₁₁, —SR₁₁, —NR₁₁R₁₁, —C(O)R₁₁, (O)NR₁₁R₁₁, —CN, —NR₁₁C(O)R₁₁, —S(O)₂NR₁₁R₁₁, —NR₁,S(O)₂R₁₁, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
or a pharmaceutically acceptable salt thereof.

11. The pharmaceutical composition of claim 10, wherein the compound of formula III is selected from:
N-[(3R)-1-azabicyclo[2.2.2]oct-3-yl]-2,3-dihydrofuro[2,3-c]pyridine-5-carboxamide;
N-[(3R)-1-azabicyclo[2.2.2]oct-3-yl]furo[2,3-c]pyridine-5-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-3-methylfuro[2,3-c]pyridine-5-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
N-[(3R)-1-Azabicyclo[2.2.2]oct-3-yl]furo[3,2-c]pyridine-6-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]thieno[2,3-c]pyridine-5-carboxamide;
N-[(2S,3R)-2-methyl-1-azabicyclo[2.2.2]oct-3-yl]thieno[3,2-c]pyridine-6-carboxamide;
N-[(3S)-1-azabicyclo[2.2.2]oct-3-yl]furo[2,3-c]pyridine-5-carboxamide; and
N-[(+/−)1-azabicyclo[2.2.2]oct-3-yl]furo[2,3-c]pyridine-5-carboxamide;
and pharmaceutically acceptable salts thereof.

12. The pharmaceutical composition of claim 1, wherein the first therapeutic agent is ziprasidone or a pharmaceutically acceptable salt thereof and the second therapeutic agent is a compound of formula IV 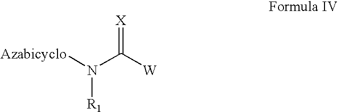
wherein Azabicyclo is 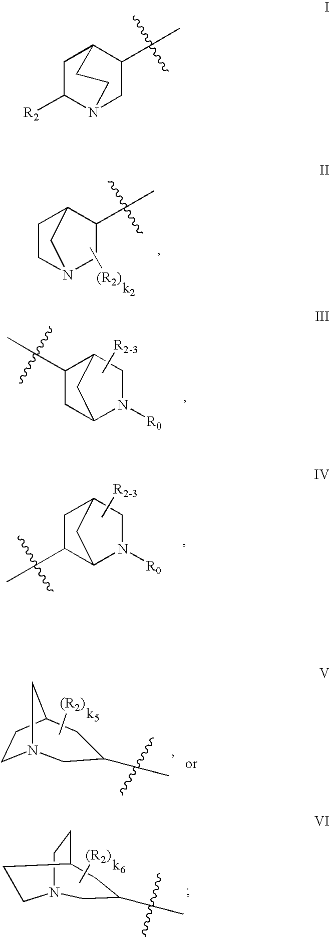
W is 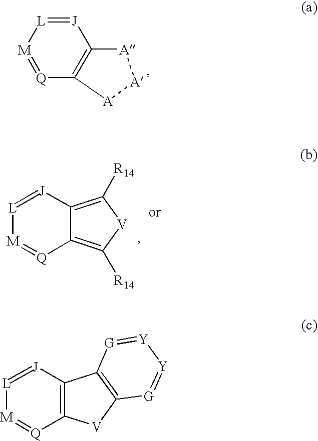
provided that the bond between the —C(═X)— group and the W group may be attached at any available carbon atom within the W group as provided in R₃, R₆, and R₁₅;
X is O, or S;
R₀ is H, lower alkyl, substituted lower alkyl, or halogenated lower alkyl;
each R₁ is H, alkyl, cycloalkyl, halogenated alkyl, substituted phenyl, or substituted naphthyl;
each R₂ is alkyl, halogenated alkyl, substituted alkyl, cycloalkyl, aryl, F, Cl, Br, I, or R₂ is absent provided that k₂, k₅, or k₆ is 0;
R₂₋₃ is H, alkyl, substituted alkyl, halogenated alkyl, F, Cl, Br, or I;
k₂ is 0 or 1;
k₅ and k₆ are independently 0, 1, or 2;
A---A′---A″ is N(R₄)—C(R₃)═C(R₃), N═C(R₃)—C(R₁₅)₂, C(R₃)═C(R₃)—N(R₄), C(R₃)₂—N(R₄)—C(R₃)₂, C(R₁₅)₂—C(R₃)═N, N(R₄)—C(R₃)₂—C(R₃)₂,
C(R₃)₂—C(R₃)₂—N(R₄), O—C(R₃)═C(R₃), O—C(R₃)₂—C(R₃)₂, C(R₃)₂—O—C(R₃)₂, C(R₃)═C(R₃)—O, C(R₃)₂—C(R₃)₂—O, S—C(R₃)═C(R₃), S—C(R₃)₂—C(R₃)₂,
C(R₃)₂—S—C(R₃)₂, C(R₃)═C(R₃)—S, or C(R₃)₂—C(R₃)₂—S;
each R₃ is independently a bond to the core molecule provided that only one R₃ and no R₆ or R₁₅ is also said bond, H, alkyl, substituted alkyl, halogenated alkyl, alkenyl, substituted alkenyl, halogenated alkenyl, alkynyl, substituted alkynyl, halogenated alkynyl, —CN, —NO₂, F, Br, Cl, I, —OR₁₉, —C(O)N(R₁₀)₂, —N(R₁₀)₂, —SR₁₉,
—S(O)₂R₁₉, —C(O)R₁₉, —CO₂R₁₉, aryl, R₇, or R₉;
J, L, M, and Q are N or C(R₆) provided that only one of J, L, M, or Q, is N and the others are C(R₆), further provided that when the core molecule is attached to the pyridinyl moiety at M, Q is C(H), and further provided that there is only one attachment to the core molecule;
G and Y are C(R₆), provided that when the molecule is attached to the phenyl moiety at Y, G is CH;
R₄ is H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, or R₉;
each R₅ is independently H, lower alkyl, or lower alkenyl;
each R₆ is independently H, F, Br, I, Cl, —CN, —CF₃, —OR₅, —SR₅, —N(R₅)₂, or a bond to the core molecule provided that only one R₆ and no R₃ or R₁₅ is said bond;
V is selected from O, S, or N(R₄);
R₇ is 5-membered heteroaromatic mono-cyclic moieties containing within the ring 1-3 heteroatoms independently selected from the group consisting of ═N—, —N(R₁₇)—, —O—, and —S—, and having 0-1 substituent selected from R₁₈ and further having 0-3 substituents independently selected from F, Cl, Br, or I, or R₇ is 9-membered fused-ring moieties having a 6-membered ring fused to a 5-membered ring including the formula 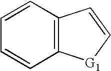
wherein G₁ is O, S or NR₁₇, 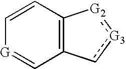
wherein G is C(R₁₆) or N, and each G₂ and G₃ are independently selected from C(R₁₆)₂, C(R₁₆), O, S, N, and N(R₁₈), provided that both G₂ and G₃ are not simultaneously O, simultaneously S, or simultaneously O and S, or 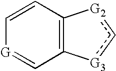
wherein G is C(R₁₆) or N, and each G₂ and G₃ are independently selected from C(R₁₆)₂, C(R₁₆), O, S, N, and N(R₁₇), each 9-membered fused-ring moiety having 0-1 substituent selected from R₁₈ and further having 0-3 substituent(s) independently selected from F, Cl, Br, or I, wherein the R₇ moiety attaches to other substituents as defined in formula I at any position on either ring as valency allows;
each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl, or substituted phenyl;
R₉ is 6-membered heteroaromatic mono-cyclic moieties containing within the ring 1-3 heteroatoms selected from ═N— and having 0-1 substituent selected from R₁₈ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₁₈ and 0-3 substituent(s) independently selected from F, Cl, Br, or I, and having a bond directly or indirectly attached to the core molecule where valency allows;
each R₁₀ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, phenyl, or substituted phenyl;
each R₁₁ is independently H, alkyl, cycloalkyl, heterocycloalkyl, halogenated alkyl, halogenated cycloalkyl, or halogenated heterocycloalkyl;
R₁₂ is —NO₂, —CN, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁,
—C(O)NR₁₁R₁₁, —NR₁₁C(O)R₁₁, —S(O)₂NR₁₁R₁₁, or —NR₁₁S(O)₂R₁₁;
R₁₃ is —CN, —CF₃, —NO₂, —OR₁₁, —SR₁₁, —NR₁₁R₁₁, —C(O)R₁₁, —C(O)NR₁₁R₁₁,
—NR₁₁C(O)R₁₁, —S(O)₂NR₁₁R₁₁, or —NR₁₁S(O)₂R₁₁;
each R₁₄ is H, alkyl, substituted alkyl, halogenated alkyl, alkenyl, substituted alkenyl, halogenated alkenyl, alkynyl, substituted alkynyl, halogenated alkynyl, F, Br, Cl, I, —CN, —NO₂, —OR₁₉, —C(O)N(R₁₀)₂, —N(R₁₀)₂, —SR₁₉, —S(O)₂R₁₉, —C(O)R₁₉,
—CO₂R₁₉, aryl, R₇ or R₉;
each R₁₅ is independently alkyl, substituted alkyl, halogenated alkyl, alkenyl, substituted alkenyl, halogenated alkenyl, alkynyl, substituted alkynyl, halogenated alkynyl, F, Br, Cl, I, —CN, —NO₂, —OR₁₉, —C(O)N(R₁₀)₂, —N(R₁₀)₂, —SR₁₉, —CO₂R₁₉, aryl, R₇, R₉, or a bond to the core molecule provided that only one R₁₅ and no R₆ or R₃ is said bond;
each R₁₆ is independently H, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, F, Cl, Br, I, —NO₂, —CN, —OR₁₁,
—SR₁₁, —NR₁₁R₁₁, —C(O)R₁₁, —C(O)NR₁₁R₁₁, —NR₁₁C(O)R₁₁, —S(O)₂NR₁₁R₁₁,
—NR₁₁S(O)₂R₁₁, or a bond directly or indirectly attached to the core molecule, provided that there is only one said bond to the core molecule within the 9-membered fused-ring moiety, further provided that the fused-ring moiety has 0-1 substituent selected from alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, or —NR₁₁S(O)₂R₁₁, and further provided that the fused-ring moiety has 0-3 substituent(s) selected from F, Cl, Br, or I;
R₁₇ is H, alkyl, halogenated alkyl, substituted alkyl, cycloalkyl, halogenated cycloalkyl, substituted cycloalkyl, phenyl, —SO₂R₈, or phenyl having 1 substituent selected from R₁₈ and further having 0-3 substituents independently selected from F, Cl, Br, or I;
R₁₈ is alkyl, cycloalkyl, heterocycloalkyl, halogenated alkyl, halogenated cycloalkyl, halogenated heterocycloalkyl, —OR₁₁, —SR₁₁, —NR₁₁R₁₁, —C(O)R₁₁,
—C(O)NR₁₁R₁₁, —CN, —NR₁₁C(O)R₁₁, —S(O)₂NR₁₁R₁₁, —NR₁₁S(O)₂R₁₁, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
R₁₉ is H, alkyl, cycloalkyl, substituted alkyl, halogenated alkyl, substituted phenyl, or substituted naphthyl;
or a pharmaceutically acceptable salt thereof.

13. The pharmaceutical composition of claim 12, wherein the compound of formula IV is selected from:
Exo-4(S)-N-(1-azabicyclo[2.2.1]hept-3-yl)furo[2,3-c]pyridine-5-carboxamide;
N-((3R,5R)-1-azabicyclo[3.2.1]oct-3-yl)furo[2,3-c]pyridine-5-carboxamide;
N-[(exo-1-azabicyclo[2.2.1]hept-3-yl]furo[3,2-c]pyridine-6-carboxamide;
N-((3R,5R)-1-azabicyclo[3.2.1]oct-3-yl)furo[3,2-c]pyridine-6-carboxamide;
Exo-4(S)-N-(1-azabicyclo[2.2.1]hept-3-yl)-thieno[2,3-c]pyridine-5-carboxamide;
N-((3R,5R)-1-azabicyclo[3.2.1]oct-3-yl)-thieno[2,3-c]pyridine-5-carboxamide;
Exo-4(S)-N-(1-azabicyclo[2.2.1]hept-3-yl)-thieno[3,2-c]pyridine-6-carboxamide; and
N-((3R,5R)-1-azabicyclo[3.2.1]oct-3-yl)-thieno[3,2-c]pyridine-6-carboxamide;
and pharmaceutically acceptable salts thereof.

14. A method for treating a cognitive impairment disorder in a subject in need thereof comprising administering to said subject
a) an amount of a first therapeutic agent which is an atypical antipsychotic; and
b) an amount of a second therapeutic agent which is a nicotinic receptor agonist or antagonist;
wherein the amounts of (a) and (b) are together effective in treating said cognitive impairment disorder.

15. The method of claim 14 wherein the first therapeutic agent is ziprasidone or a pharmaceutically acceptable salt thereof.

16. The method of claim 14 where the first therapeutic agent is selected from the group consisting of olanzapine, aripiprazole, clozapine, risperidone, sertindole, quetiapine, amisulpride, ziprasidone, asenapine, paliperidone, bifeprunox, and pharmaceutically acceptable salts of any of the foregoing.

17. The method of claim 14, wherein (a) and (b) are administered to the subject in a single pharmaceutical composition additionally comprising a pharmaceutically acceptable vehicle, carrier or diluent, by a method selected from the group consisting of oral, transmucosal, transdermal, nasal, pulmonary, buccal, parenteral rectal, and sublingual.

18. The method of claim 17, wherein parenteral administration to the subject is selected from the group consisting of intravenous, intramuscular, subcutaneous, and intradermal.

19. The method of claim 14, wherein said cognitive impairment disorder is selected from the group consisting of Alzheimer's disease, age related memory disorder, dementia, including vascular dementia, cognitive impairment caused by traumatic brain injury, dementia due to other general medical conditions, substance-induced persisting dementia, dementia due to multiple etiologies, dementia not otherwise specified, and cognitive disorder not otherwise specified.

20. The method of claim 15, wherein the ziprasidone or ziprasidone salt is administered at a dosage of between about 5 mg to about 460 mg daily.

21. The method of claim 20, wherein the ziprasidone or ziprasidone salt is administered at a dosage of between about 10 mg to about 320 mg daily.

22. The method of claim 21, wherein the ziprasidone or ziprasidone salt is administered at a dosage of between about 20 mg to about 200 mg daily.

23. The method of claim 22, wherein the ziprasidone or ziprasidone salt is administered at a dosage of between about 20 mg to about 160 mg daily.

24. A method for treating a psychotic disorder or condition in a subject in need thereof comprising administering to said subject
a) an amount of a first therapeutic agent which is an atypical antipsychotic; and
b) an amount of a second therapeutic agent which is a nicotinic receptor agonist or antagonist;
wherein the amounts of (a) and (b) are together effective in treating said psychotic disorder or condition.

25. The method of claim 24, wherein the first therapeutic agent is ziprasidone or a pharmaceutically acceptable salt of ziprasidone.

26. The method of claim 24, wherein the first therapeutic agent is ziprasidone, olanzapine, aripiprazole, clozapine, risperidone, sertindole, quetiapine, amisulpride, asenapine, paliperidone, bifeprunox, and pharmaceutically acceptable salts of any of the foregoing.

27. The method of claim 24, wherein (a) and (b) are administered to the subject in a single pharmaceutical composition additionally comprising a pharmaceutically acceptable vehicle, carrier or diluent, by a method selected from the group consisting of oral, transmucosal, transdermal, nasal, pulmonary, buccal, parenteral, rectal, and sublingual.

28. The method of claim 27, wherein parenteral administration to the subject is selected from the group consisting of intravenous, intramuscular, subcutaneous, and intradermal.

29. The method of claim 24, wherein said psychotic disorder or condition is selected from the group consisting of schizophrenia, schizophreniform disorder, schizoaffective disorder, delusional disorder, brief psychotic disorder, treatment-resistant shared psychotic disorder, psychotic disorder due to a medical condition, and psychotic disorder not otherwise specified.

30. The method of claim 24, wherein the ziprasidone or ziprasidone salt is administered at a dosage of between about 10 mg to about 460 mg daily.

31. The method of claim 30, wherein the ziprasidone or ziprasidone salt is administered at a dosage of between about 20 mg to about 320 mg daily.

32. The method of claim 31, wherein the ziprasidone or ziprasidone salt is administered at a dosage of between about 40 mg to about 200 mg daily.

33. The method of claim 32, wherein the ziprasidone or ziprasidone salt is administered at a dosage of between about 20 mg to about 160 mg daily.

34. The method of claim 14, wherein said nicotinic receptor antagonist is selected from the group consisting of mecamylamine, amantadine, pempidine, di-hydro-beta-erythroidine, hexamethonium, erysodine, methyllycaconitine, chlorisondamine, trimethaphan, normecamylamine, N-(1,2,2)trimethyl-1-bicyclo[2,2,1,]-heptylbenzenamine, dimethylaminoisocamphane, exoaminonorbornane, 2,2,6,6-tetramethylpiperidine, 2,2,6,6-tetramethyl-4-aminopiperidine, erysodine, a phenyltropane carboxylic acid methyl ester, a arylpempidine analogue, ibogaine, pharmaceutically acceptable salts of any of the foregoing, and prodrugs of any of the foregoing, and pharmaceutically acceptable salts of said prodrugs; and said nicotine receptor agonist is selected from the group consisting of varenicline tartrate, a gamma nicotine compound, an alpha nicotine compound, fluorine containing derivatives of nicotine, N-lower alkyl analogs of nicotine, nicotine compounds in the (R)-(+)-form, a pyridyalkylpiperidine or pyridylalkylpyrrolidine compound, and a nicotine agonist derivative.

35. The method of claim 15, wherein said nicotinic receptor antagonist is selected from the group consisting of mecamylamine, amantadine, pempidine, di-hydro-beta-erythroidine, hexamethonium, erysodine, methyllycaconitine, chlorisondamine, trimethaphan, normecamylamine, N-(1,2,2)trimethyl-1-bicyclo[2,2,1,]-heptylbenzenamine, dimethylaminoisocamphane, exoaminonorbornane, 2,2,6,6-tetramethylpiperidine, 2,2,6,6-tetramethyl-4-aminopiperidine, erysodine, a phenyltropane carboxylic acid methyl ester, a arylpempidine analogue, ibogaine, pharmaceutically acceptable salts of any of the foregoing, and prodrugs of any of the foregoing, and pharmaceutically acceptable salts of said prodrug; and said nicotine receptor agonist is selected from the group consisting of varenicline tartrate, a gamma nicotine compound, an alpha nicotine compound, fluorine containing derivatives of nicotine, N-lower alkyl analogs of nicotine, nicotine compounds in the (R)-(+)-form, a pyridyalkylpiperidine or pyridylalkylpyrrolidine compound, and a nicotine agonist derivative.

36. The method of claim 24, wherein said nicotinic receptor antagonist is selected from the group consisting of mecamylamine, amantadine, pempidine, di-hydro-beta-erythroidine, hexamethonium, erysodine, methyllycaconitine, chlorisondamine, trimethaphan, normecamylamine, N-(1,2,2)trimethyl-1-bicyclo[2,2,1,]-heptylbenzenamine, dimethylaminoisocamphane, exoaminonorbornane, 2,2,6,6-tetramethylpiperidine, 2,2,6,6-tetramethyl-4-aminopiperidine, erysodine, a phenyltropane carboxylic acid methyl ester, a arylpempidine analogue, ibogaine, pharmaceutically acceptable salts of any of the foregoing, prodrugs of any of the foregoing, and pharmaceutically acceptable salts of said prodrugs; and said nicotine receptor agonist is selected from the group consisting of varenicline tartrate, a gamma nicotine compound, an alpha nicotine compound, fluorine containing derivatives of nicotine, N-lower alkyl analogs of nicotine, nicotine compounds in the (R)-(+)-form, a pyridyalkylpiperidine or pyridylalkylpyrrolidine compound, and a nicotine agonist derivative.

37. The method of claim 25, wherein said nicotinic receptor antagonist is selected from the group consisting of mecamylamine, amantadine, pempidine, di-hydro-beta-erythroidine, hexamethonium, erysodine, methyllycaconitine, chlorisondamine, trimethaphan, normecamylamine, N-(1,2,2)trimethyl-1-bicyclo[2,2,1,]-heptylbenzenamine, dimethylaminoisocamphane, exoaminonorbornane, 2,2,6,6-tetramethylpiperidine, 2,2,6,6-tetramethyl-4-aminopiperidine, erysodine, a phenyltropane carboxylic acid methyl ester, a arylpempidine analogue, ibogaine, pharmaceutically acceptable salts of any of the foregoing, prodrugs of any of the foregoing, and pharmaceutically acceptable salts of said prodrugs; and said nicotine receptor agonist is selected from the group consisting of varenicline tartrate, a gamma nicotine compound, an alpha nicotine compound, fluorine containing derivatives of nicotine, N-lower alkyl analogs of nicotine, nicotine compounds in the (R)-(+)-form, a pyridyalkylpiperidine or pyridylalkylpyrrolidine compound, and a nicotine agonist derivative.

38. The method of claim 26, wherein said nicotinic receptor antagonist is selected from the group consisting of mecamylamine, amantadine, pempidine, di-hydro-beta-erythroidine, hexamethonium, erysodine, methyllycaconitine, chlorisondamine, trimethaphan, normecamylamine, N-(1,2,2)trimethyl-1-bicyclo[2,2,1,]-heptylbenzenamine, dimethylaminoisocamphane, exoaminonorbornane, 2,2,6,6-tetramethylpiperidine, 2,2,6,6-tetramethyl-4-aminopiperidine, erysodine, a phenyltropane carboxylic acid methyl ester, a arylpempidine analogue, ibogaine, pharmaceutically acceptable salts of any of the foregoing, prodrugs of any of the foregoing, and pharmaceutically acceptable salts of said prodrugs; and said nicotine receptor agonist is selected from the group consisting of varenicline tartrate, a gamma nicotine compound, an alpha nicotine compound, fluorine containing derivatives of nicotine, N-lower alkyl analogs of nicotine, nicotine compounds in the (R)-(+)-form, a pyridyalkylpiperidine or pyridylalkylpyrrolidine compound, and a nicotine agonist derivative.

39. The method of claim 15, wherein the second therapeutic agent is varenicline or a pharmaceutically acceptable salt thereof.

40. The method of claim 25, wherein the second therapeutic agent is varenicline or a pharmaceutically acceptable salt thereof.

41. The method of claim 25 wherein the second therapeutic agent is an alpha 7 subtype selective nicotinic receptor agonist.

42. The method of claim 25, wherein the second therapeutic agent is a compound of formula II 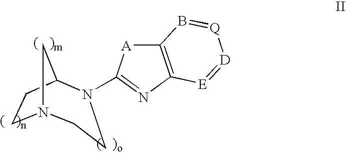
wherein
n=1-2;
m=1-2;
o=1-2;
A=O, S or NR¹;
B=N or CR²;
Q=N or CR³;
D=N or CR⁴;
E=N or CR⁵;
R¹ is H, a straight chain or branched (C₁-C₈)alkyl, C(═O)OR⁶, CH₂R⁶, C(═O)NR⁶R⁷, C(═O)R⁶, or SO₂R⁶;
each R², R³, R⁴ and R⁵ is independently selected from F, Cl, Br, I, nitro, cyano, CF₃, —NR⁶R⁷, —NR⁶C(═O)R⁷, —NR⁶C(═O)NR⁷R⁸, —NR⁶C(═O)OR⁷, —NR⁶S(═O)₂R⁷, —NR⁶S(═O)₂NR⁷R⁸, —OR⁶, —OC(═O)R⁶, —OC(═O)OR⁶, —OC(═O)NR⁶R⁷, —OC(═O)SR⁶, —C(═O)OR⁶, —C(═O)R⁶, —C(═O)NR⁶R⁷, —SR⁶, —S(═O)R⁶, —S(═O)₂R⁶, —S(═O)₂NR⁶R⁷, and R⁶;
each R⁶, R⁷, and R⁸ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, 5-11 membered heterobicycloalkenyl, (C₆-C₁₁)aryl, and 5-12 membered heteroaryl; wherein each R⁶, R⁷, and R⁸ is optionally substituted with from one to six substituents, independently selected from F, Cl, Br, I, nitro, cyano, CF₃, —NR⁹R¹⁰, —NR⁹C(═O)R¹⁰, —NR⁹C(═O)NR¹⁰R¹¹, —NR⁹C(═O)OR¹⁰, —NR⁹S(═O)₂R¹⁰, —NR⁹S(═O)₂NR¹⁰R¹¹, —OR⁹, —OC(═O)R⁹, —OC(═O)OR⁹, —OC(═O)NR⁹R¹⁰, —OC(═O)SR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹;
each R⁹, R¹⁰ and R¹¹ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, (5-11 membered) heterobicycloalkenyl, (C₆-C₁₁)aryl or 5-12 membered heteroaryl; wherein each R⁹, R¹⁰ and R¹¹ is optionally substituted with from one to six substituents independently selected from F, Cl, Br, I, nitro, cyano, CF₃, —NR¹²R¹³, —NR¹²C(═O)R¹³, —NR¹²C(═O)NR¹³R¹⁴, —NR¹²C(═O)OR¹³, —NR¹²S(═O)₂R¹³, —NR¹²S(═O)₂NR¹³R¹⁴, —OR¹², —OC(═O)R¹², —OC(═O)OR¹², —OC(═O)NR¹²R¹³, —OC(═O)SR¹², —C(═O)OR¹², —C(═O)R¹², —C(═O)NR¹²R¹³, —SR¹², —S(═O)R¹², —S(═O)₂R¹², —S(═O)₂NR¹²R¹³ and R¹²;
each R¹², R¹³, and R¹⁴ is independently selected from H, straight chain or branched (C₁-C₈)alkyl, straight chain or branched (C₂-C₈)alkenyl, straight chain or branched (C₂-C₈)alkynyl, (C₃-C₈)cycloalkyl, (C₄-C₈)cycloalkenyl, 3-8 membered heterocycloalkyl, (C₅-C₁₁)bicycloalkyl, (C₇-C₁₁)bicycloalkenyl, 5-11 membered heterobicycloalkyl, 5-11 membered heterobicycloalkenyl, (C₆-C₁₁)aryl and (5-12 membered) heteroaryl;
or R² and R³, or R³ and R⁴, or R⁴ and R⁵, may form another 6-membered aromatic or heteroaromatic ring sharing B and Q, or Q and D, or D and E, respectively, and may be optionally substituted with from one to four substitutuents independently selected from the group of radicals set forth in the definition of R⁶, R⁷ and R⁸ above;
or a pharmaceutically acceptable salts thereof.

43. The method of claim 42, wherein the compound of formula II is selected from:
4-oxazolo[5,4-b]pyridin-2-yl-1,4-diazabicyclo[3.2.2]nonane;
4-oxazolo[5,4-c]pyridin-2-yl-1,4-diazabicyclo[3.2.2]nonane;
4-oxazolo[4,5-c]pyridin-2-yl-1,4-diazabicyclo[3.2.2]nonane;
4-oxazolo[4,5-b]pyridin-2-yl-1,4-diazabicyclo[3.2.2]nonane;
4-(5-methyl-oxazolo[4,5-b]pyridin-2-yl)-1,4-diazabicyclo[3.2.2]nonane;
4-(6-phenyl-oxazolo[5,4-b]pyridin-2-yl)-1,4-diazabicyclo[3.2.2]nonane;
4-(6-bromo-oxazolo[4,5-b]pyridin-2-yl)-1,4-diazabicyclo[3.2.2]nonane; and
4-(6-phenyl-oxazolo[4,5-b]pyridin-2-yl)-1,4-diazabicyclo[3.2.2]nonane;
and pharmaceutically acceptable salts thereof.

44. The method claim 25, wherein the second therapeutic agent is a compound of formula III 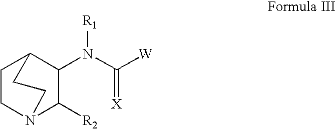
wherein W is 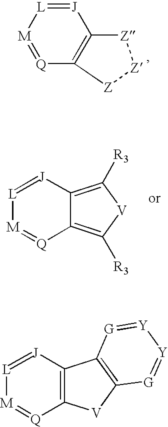
provided that the bond between the —C(═X)— group and the W group may be attached at any available carbon atom within the W group as provided in R₃, R₆, and R₁₅;
X is O, or S;
each R₁ is H, alkyl, cycloalkyl, halogenated alkyl, substituted phenyl, or substituted naphthyl;
R₂ is H, alkyl, halogenated alkyl, substituted alkyl, cycloalkyl, or aryl;
Z---Z′---Z″ is selected from N(R₄)—C(R₃)═C(R₃), N═C(R₃)—C(R₁₅)₂, C(R₃)═C(R₃)—N(R₄), C(R₃)₂—N(R₄)—C(R₃)₂, C(R₁₅)₂—C(R₃)═N, N(R₄)—C(R₃)₂—C(R₃)₂, C(R₃)₂—C(R₃)₂—N(R₄), O—C(R₃)═C(R₃), O—C(R₃)₂—C(R₃)₂, C(R₃)₂—O—C(R₃)₂, C(R₃)═C(R₃)—O, C(R₃)₂—C(R₃)₂—O, S—C(R₃)═C(R₃), S—C(R₃)₂—C(R₃)₂, C(R₃)₂—S—C(R₃)₂, C(R₃)═C(R₃)—S, or C(R₃)₂—C(R₃)₂—S;
each R₃ is independently a bond to the core molecule provided that only one R₃ and no R₆ or R₁₅ is also said bond, H, F, Br, Cl, I, alkyl, substituted alkyl, halogenated alkyl, alkenyl, substituted alkenyl, halogenated alkenyl, alkynyl, substituted alkynyl, halogenated alkynyl, heterocycloalkyl, substituted heterocycloalkyl, lactam heterocycloalkyl, —CN, —NO₂, —OR₁, —C(O)N(R₁₀)₂,
—NR₁COR₁₆, —N(R₁₀)₂, —SR₁, —S(O)₂R₁, —C(O)R₁₆, —CO₂R₁, aryl, R₇, or R₉;
J, L, M, and Q are N or C(R₆) provided that only one of J, L, M, or Q, is N and the others are C(R₆), further provided that when the core molecule is attached to the pyridinyl moiety at M, Q is C(H), and further provided that there is only one attachment to the core molecule;
G and Y are C(R₆), provided that when the molecule is attached to the phenyl moiety at Y, G is CH;
R₄ is H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, or R₉;
each R₅ is independently H, C₁₋₃ alkyl, or C₂₋₄ alkenyl;
each R₆ is independently H, F, Br, I, Cl, —CN, —CF₃, —OR₅, —SR₅, or —N(R₅)₂, or a bond to the core molecule provided that only one P6 and no R₃ or R₁₅ is said bond,
V is selected from O, S, or N(R₄);
R₇ is 5-membered heteroaromatic mono-cyclic moieties containing within the ring 1-3 heteroatoms independently selected from the group consisting of —O—, ═N—, —N(R₁₉)—, and —S—, and having 0-1 substituent selected from R₂₀ and further having 0-3 substituents independently selected from F, Cl, Br, or I, or R₇ is a 9-membered fused-ring moiety having a 6-membered ring fused to a 5-membered ring and having the formula 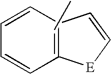
wherein E is O, S, or NR₁₉, 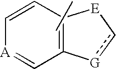
wherein E and G are independently selected from CR₁₈, O, S, N, or NR₁₉, and A is CR₁₈ or N, or 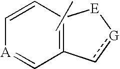
wherein E and G are independently selected from CR₁₈, O, S, N, or NR₁₉, and A is CR₁₈ or N, each 9-membered fused-ring moiety having 0-1 substituent selected from R₂₀ and further having 0-3 substituent(s) independently selected from F, Cl, Br, or I, and having a bond directly or indirectly attached to the core molecule where valency allows in either the 6-membered or the 5-membered ring of the fused-ring moiety;
each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl, or substituted phenyl;
R₉ is 6-membered heteroaromatic mono-cyclic moieties containing within the ring 1-3 heteroatoms selected from ═N— and having 0-1 substituent selected from R₂₀ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₂₀ and 0-3 substituent(s) independently selected from F, Cl, Br, or I and having a bond directly or indirectly attached to the core molecule where valency allows;
each R₁₀ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, phenyl, or substituted phenyl;
each R₁₁ is independently H, alkyl, cycloalkyl, heterocyclo-alkyl, halogenated alkyl, halogenated cycloalkyl, or halogenated heterocycloalkyl;
R₁₃ is —OR₁₁, —SR₁₁, —NR₁₁R₁₁, —C(O)R₁₁, —C(O)NR₁₁R₁₁, —CN, —CF₃, —NR₁₁C(O)R₁₁, —S(O)₂NR₁₁R₁₁, —NR₁₁S(O)₂R₁₁, or —NO₂;
each R₁₅ is independently a bond to the core molecule provided that only one R₁₅ and no R₆ or R₃ is also said bond, H, F, Br, Cl, I, alkyl, substituted alkyl, halogenated alkyl, alkenyl, substituted alkenyl, halogenated alkenyl, alkynyl, substituted alkynyl, halogenated alkynyl, heterocycloalkyl, substituted heterocycloalkyl, lactam heterocycloalkyl, —CN, —NO₂, —OR₁, —C(O)N(R₁₀)₂, —NR₁COR₁₆, —N(R₁₀)₂, —SR₁, —CO₂R₁, aryl, R₇, or R₉;
R₁₆ is H, alkyl, substituted alkyl, cycloalkyl, halogenated alkyl, heterocycloalkyl, substituted heterocycloalkyl, substituted phenyl, or substituted naphthyl;
each R₁₈ is independently H, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, —NR₁₁S(O)₂R₁₁, F, Cl, Br, I, or a bond directly or indirectly attached to the core molecule, provided that there is only one said bond to the core molecule within the 9-membered fused-ring moiety, further provided that the fused-ring moiety has 0-1 substituent selected from alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, or —NR₁₁S(O)₂R₁₁, and further provided that the fused-ring moiety has 0-3 substituent(s) selected from F, Cl, Br, or I;
R₁₉ is H, alkyl, halogenated alkyl, substituted alkyl, cycloalkyl, halogenated cycloalkyl, substituted cycloalkyl, phenyl, —SO₂R₈, or phenyl having 1 substituent selected from R₂₀ and further having 0-3 substituents independently selected from F, Cl, Br, or I;
R₂₀ is alkyl, cycloalkyl, heterocycloalkyl, halogenated alkyl, halogenated cycloalkyl, halogenated heterocycloalkyl, —OR₁₁, —SR₁₁, —NR₁₁R₁₁, —C(O)R₁₁, —C(O)NR₁₁R₁₁, —CN, —NR₁₁C(O)R₁₁, —S(O)₂NR₁₁R₁₁, —NR₁₁S(O)₂R₁₁, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, 1, or R₁₃;
or a pharmaceutically acceptable salt thereof.

45. The method of claim 44, wherein the compound of formula III is selected from:
N-[(3R)-1-azabicyclo[2.2.2]oct-3-yl]-2,3-dihydrofuro[2,3-c]pyridine-5-carboxamide;
N-[(3R)-1-azabicyclo[2.2.2]oct-3-yl]furo[2,3-c]pyridine-5-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-3-methylfuro[2,3-c]pyridine-5-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
N-[(3R)-1-Azabicyclo[2.2.2]oct-3-yl]furo[3,2-c]pyridine-6-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]thieno[2,3-c]pyridine-5-carboxamide;
N-[(2S,3R)-2-methyl-1-azabicyclo[2.2.2]oct-3-yl]thieno[3,2-c]pyridine-6-carboxamide;
N-[(3S)-1-azabicyclo[2.2.2]oct-3-yl]furo[2,3-c]pyridine-5-carboxamide; and
N-[(+/−)1-azabicyclo[2.2.2]oct-3-yl]furo[2,3-c]pyridine-5-carboxamide;
and pharmaceutically acceptable salts thereof.

46. The method of claim 25, wherein the second therapeutic agent is a compound of formula IV 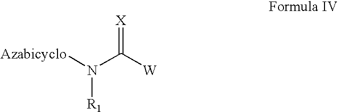
wherein Azabicyclo is 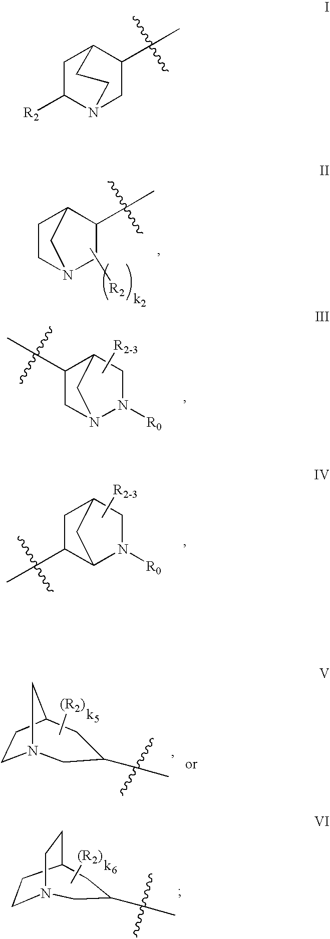
W is 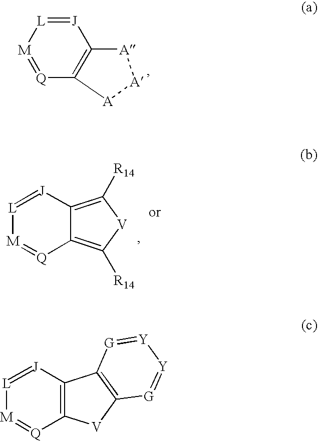
provided that the bond between the —C(═X)— group and the W group may be attached at any available carbon atom within the W group as provided in R₃, R₆, and R₁₅;
X is O, or S;
R₀ is H, lower alkyl, substituted lower alkyl, or halogenated lower alkyl;
each R₁ is H, alkyl, cycloalkyl, halogenated alkyl, substituted phenyl, or substituted naphthyl;
each R₂ is alkyl, halogenated alkyl, substituted alkyl, cycloalkyl, aryl, F, Cl, Br, I, or R₂ is absent provided that k₂, k₅, or k₆ is 0;
R₂₋₃ is H, alkyl, substituted alkyl, halogenated alkyl, F, Cl, Br, or I;
k₂ is 0 or 1;
k₅ and k₆ are independently 0, 1, or 2;
A---A′---A″ is N(R₄)—C(R₃)═C(R₃), N═C(R₃)—C(R₁₅)₂, C(R₃)═C(R₃)—N(R₄), C(R₃)₂—N(R₄)—C(R₃)₂, C(R₁₅)₂—C(R₃)═N, N(R₄)—C(R₃)₂—C(R₃)₂,
C(R₃)₂—C(R₃)₂—N(R₄), O—C(R₃)═C(R₃), O—C(R₃)₂—C(R₃)₂, C(R₃)₂—O—C(R₃)₂, C(R₃)═C(R₃)—O, C(R₃)₂—C(R₃)₂—O, S—C(R₃)═C(R₃), S—C(R₃)₂—C(R₃)₂,
C(R₃)₂—S—C(R₃)₂, C(R₃)═C(R₃)—S, or C(R₃)₂—C(R₃)₂—S;
each R₃ is independently a bond to the core molecule provided that only one R₃ and no R₆ or R₁₅ is also said bond, H, alkyl, substituted alkyl, halogenated alkyl, alkenyl, substituted alkenyl, halogenated alkenyl, alkynyl, substituted alkynyl, halogenated alkynyl, —CN, —NO₂, F, Br, Cl, I, —OR₁₉, —C(O)N(R₁₀)₂, —N(R₁₀)₂, —SR₁₉,
—S(O)₂R₁₉, —C(O)R₁₉, —CO₂R₁₉, aryl, R₇, or R₉;
J, L, M, and Q are N or C(R₆) provided that only one of J, L, M, or Q, is N and the others are C(R₆), further provided that when the core molecule is attached to the pyridinyl moiety at M, Q is C(H), and further provided that there is only one attachment to the core molecule;
G and Y are C(R₆), provided that when the molecule is attached to the phenyl moiety at Y, G is CH;
R₄ is H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, or R₉;
each R₅ is independently H, lower alkyl, or lower alkenyl;
each R₆ is independently H, F, Br, I, Cl, —CN, —CF₃, —OR₅, —SR₅, —N(R₅)₂, or a bond to the core molecule provided that only one R₆ and no R₃ or R₁₅ is said bond;
V is selected from O, S, or N(R₄);
R₇ is 5-membered heteroaromatic mono-cyclic moieties containing within the ring 1-3 heteroatoms independently selected from the group consisting of ═N—, —N(R₁₇)—, —O—, and —S—, and having 0-1 substituent selected from R₁₈ and further having 0-3 substituents independently selected from F, Cl, Br, or I, or R₇ is 9-membered fused-ring moieties having a 6-membered ring fused to a 5-membered ring including the formula 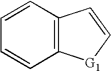
wherein G₁ is O, S or NR₁₇, 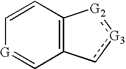
wherein G is C(R₁₆) or N, and each G₂ and G₃ are independently selected from C(R₁₆)₂, C(R₁₆), O, S, N, and N(R₁₈), provided that both G₂ and G₃ are not simultaneously O, simultaneously S, or simultaneously O and S, or 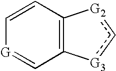
wherein G is C(R₁₆) or N, and each G₂ and G₃ are independently selected from C(R₁₆)₂, C(R₁₆), O, S, N, and N(R₁₇), each 9-membered fused-ring moiety having 0-1 substituent selected from R₁₈ and further having 0-3 substituent(s) independently selected from F, Cl, Br, or I, wherein the R₇ moiety attaches to other substituents as defined in formula I at any position on either ring as valency allows;
each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl, or substituted phenyl;
R₉ is 6-membered heteroaromatic mono-cyclic moieties containing within the ring 1-3 heteroatoms selected from ═N— and having 0-1 substituent selected from R₁₈ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₁₈ and 0-3 substituent(s) independently selected from F, Cl, Br, or I, and having a bond directly or indirectly attached to the core molecule where valency allows;
each R₁₀ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, phenyl, or substituted phenyl;
each R₁₁ is independently H, alkyl, cycloalkyl, heterocycloalkyl, halogenated alkyl, halogenated cycloalkyl, or halogenated heterocycloalkyl;
R₁₂ is —NO₂, —CN, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁,
—C(O)NR₁₁R₁₁, —NR₁₁C(O)R₁₁, —S(O)₂NR₁₁R₁₁, or —NR₁₁S(O)₂R₁₁;
R₁₃ is —CN, —CF₃, —NO₂, —OR₁₁, —SR₁₁, —NR₁₁R₁₁, —C(O)R₁₁, —C(O)NR₁₁R₁₁,
—NR, ₁C(O)R₁₁, —S(O)₂NR₁₁R₁₁, or —NR₁₁S(O)₂R₁₁;
each R₁₄ is H, alkyl, substituted alkyl, halogenated alkyl, alkenyl, substituted alkenyl, halogenated alkenyl, alkynyl, substituted alkynyl, halogenated alkynyl, F, Br, Cl, I, —CN, —NO₂, —OR₁₉, —C(O)N(R₁₀)₂, —N(R₁₀)₂, —SR₁₉, —S(O)₂R₁₉, —C(O)R₁₉,
—CO₂R₁₉, aryl, R₇ or R₉;
each R₁₅ is independently alkyl, substituted alkyl, halogenated alkyl, alkenyl, substituted alkenyl, halogenated alkenyl, alkynyl, substituted alkynyl, halogenated alkynyl, F, Br, Cl, I, —CN, —NO₂, —OR₁₉, —C(O)N(R₁₀)₂, —N(R₁₀)₂, —SR₁₉, —CO₂R₁₉, aryl, R₇, R₉, or a bond to the core molecule provided that only one R₁₅ and no R₆ or R₃ is said bond;
each R₁₆ is independently H, alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, F, Cl, Br, I, —NO₂, —CN, —OR₁₁,
—SR₁₁, —NR₁₁R₁₁, —C(O)R₁₁, —C(O)NR₁₁R₁, —NR₁₁C(O)R₁₁, —S(O)₂NR₁₁R₁₁,
—NR₁₁(O)₂R₁₁, or a bond directly or indirectly attached to the core molecule, provided that there is only one said bond to the core molecule within the 9-membered fused-ring moiety, further provided that the fused-ring moiety has 0-1 substituent selected from alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —NR₁₁R₁₁, —C(O)R₁₁, —NO₂, —C(O)NR₁₁R₁₁, —CN, —NR₁₁C(O)R₁₁, —S(O)₂NR₁₁R₁₁, or —NR₁₁S(O)₂R₁₁, and further provided that the fused-ring moiety has 0-3 substituent(s) selected from F, Cl, Br, or I;
R₁₇ is H, alkyl, halogenated alkyl, substituted alkyl, cycloalkyl, halogenated cycloalkyl, substituted cycloalkyl, phenyl, —SO₂R₈, or phenyl having 1 substituent selected from R₁₈ and further having 0-3 substituents independently selected from F, Cl, Br, or I;
R₁₈ is alkyl, cycloalkyl, heterocycloalkyl, halogenated alkyl, halogenated cycloalkyl, halogenated heterocycloalkyl, —OR₁₁, —SR₁₁, —NR₁₁R₁₁, —C(O)R₁₁,
—C(O)NR₁₁R₁₁, —CN, —NR₁₁C(O)R₁₁, —S(O)₂NR₁₁R₁₁, —NR₁₁S(O)₂R₁₁, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
R₁₉ is H, alkyl, cycloalkyl, substituted alkyl, halogenated alkyl, substituted phenyl, or substituted naphthyl;
or a pharmaceutically acceptable salt thereof.

47. The method of claim 46, wherein the compound of formula IV is selected from:
Exo-4(S)-N-(1-azabicyclo[2.2.1]hept-3-yl)furo[2,3-c]pyridine-5-carboxamide;
N-((3R,5R)-1-azabicyclo[3.2.1]oct-3-yl)furo[2,3-c]pyridine-5-carboxamide;
N-[(exo-1-azabicyclo[2.2.1]hept-3-yl]furo[3,2-c]pyridine-6-carboxamide;
N-((3R,5R)-1-azabicyclo[3.2.1]oct-3-yl)furo[3,2-c]pyridine-6-carboxamide;
Exo-4(S)-N-(1-azabicyclo[2.2.1]hept-3-yl)-thieno[2,3-c]pyridine-5-carboxamide;
N-((3R,5R)-1-azabicyclo[3.2.1]oct-3-yl)-thieno[2,3-c]pyridine-5-carboxamide;
Exo-4(S)-N-(1-azabicyclo[2.2.1]hept-3-yl)-thieno[3,2-c]pyridine-6-carboxamide; and
N-((3R,5R)-1-azabicyclo[3.2.1]oct-3-yl)-thieno[3,2-c]pyridine-6-carboxamide;
and pharmaceutically acceptable salts thereof.

48. A kit for treating a subject having cognitive impairment or a psychotic disorder, said kit comprising
a) a package containing a unit dosage of ziprasidone or a pharmaceutically acceptable salt of ziprasidone; and
b) a package containing a unit dosage of a nicotinic receptor agonist or antagonist.

49. The kit of claim 48, wherein said ziprasidone or ziprasidone salt is provided in more than one unit dosage, and wherein said nicotinic receptor agonist or antagonist is provided in more than one unit dosage.
